(19) United States
(12) Patent Application Publication
(10) Pub. No.: US 2013/0230519 A1
(43) Pub. Date: Sep. 5, 2013

(54) ANTI-SERUM ALBUMIN BINDING VARIANTS

(71) Applicants: Elena De Angelis, Cambridge (GB); Carolyn Enever, Cambridge (GB); Haiqun Liu, Cambridge (GB); Malgorzata Pupecka-Swider, Cambridge (GB); Oliver Schon, Cambridge (GB)

(72) Inventors: Elena De Angelis, Cambridge (GB); Carolyn Enever, Cambridge (GB); Haiqun Liu, Cambridge (GB); Malgorzata Pupecka-Swider, Cambridge (GB); Oliver Schon, Cambridge (GB)

(21) Appl. No.: 13/817,883

(22) PCT Filed: Aug. 12, 2011
(86) PCT No.: PCT/EP11/64000

§ 371 Date: Feb. 20, 2013

Related U.S. Application Data
(60) Provisional application No. 61/375,328, filed on Aug. 20, 2010.

(51) Int. Cl. C07K 16/18; C07K 14/605; C07K 14/56; C07K 16/46; A61K 47/48

(52) U.S. Cl. CPC C07K 16/18; C07K 16/468; A61K 47/48538; C07K 14/56; C07K 14/605
USPC 424/134.1; 530/387.3; 530/391.7; 435/252.33; 435/328; 424/133.1; 536/23.53; 435/320.1; 536/23.4; 424/136.1

(57) ABSTRACT

The invention relates to improved variants of the anti-serum albumin immunoglobulin single variable domain DOM7h-14-10, as well as ligands and drug conjugates comprising such variants, compositions, nucleic acids, vectors and hosts.

The invention relates to improved variants of the anti-serum albumin immunoglobulin single variable domain DOM7h-14, as well as ligands and drug conjugates comprising such variants, compositions, nucleic acids, vectors and hosts.

### BACKGROUND OF THE INVENTION

WO04003019 and WO2008/096158 disclose anti-serum albumin (SA) binding moieties, such as anti-SA immunoglobulin single variable domains (dAbs), which have therapeutically-useful half-lives. These documents disclose monomer anti-SA dAbs as well as multi-specific ligands comprising such dAbs, e.g. ligands comprising an anti-SA dAb and a dAb that specifically binds a target antigen, such as TNFR1. Binding moieties are disclosed that specifically bind serum albumins from more than one species, e.g. human/mouse cross-reactive anti-SA dAbs.

WO05118642 and WO2006/059106 disclose the concept of conjugating or associating an anti-SA binding moiety, such as an anti-SA immunoglobulin single variable domain, to a drug, in order to increase the half-life of the drug. Protein, peptide and NCE (new chemical entity) drugs are disclosed and exemplified. WO2006/059106 discloses the use of this concept to increase the half-life of insulinotropic agents, e.g., incretin hormones such as glucagon-like peptide (GLP)-1.

Reference is also made to Holt et al, “Anti-Serum albumin domain antibodies for extending the half-lives of short lived drugs”, Protein Engineering, Design & Selection, vol. 21, no 5, pp 283-288, 2008.

WO2008/096158 discloses DOM7h-14, which is a good anti-SA dAb. It would be desirable to provide improved dAbs that are variants of DOM7h-14 and that specifically bind serum albumin, preferably albumins from human and non-human species, which would provide utility in animal models of disease as well as for human therapy and/or diagnosis. It would also be desirable to provide for the choice between relatively modest- and high-affinity anti-SA binding moieties (dAbs). Such moieties could be linked to drugs, the anti-SA binding moiety being chosen according to the contemplated end-application. This would allow the drug to be better tailored to treating and/or preventing chronic or acute indications, depending upon the choice of anti-SA binding moiety. For some applications, it would be desirable to provide anti-SA dAbs, that are monomeric or substantially so in solution. This would especially be advantageous when the anti-SA dAb is linked to a binding moiety, e.g., a dAb, that specifically binds a cell-surface receptor, such as TNFR1, with the aim of antagonizing the receptor. The monomeric state of the anti-SA dAb is useful in reducing the chance of receptor cross-linking, since multimers are less likely to form which could bind and cross-link receptors (e.g. TNFR1) on the cell surface, thus increasing the likelihood of receptor agonism and detrimental receptor signaling.

### SUMMARY OF THE INVENTION

Improved anti-SA dAbs are described in PCT/EP2010/052008 and PCT/EP2010/052007.

In one aspect, the invention provides an anti-serum albumin (SA) immunoglobulin single variable domain selected from DOM7h-14-56 (SEQ ID NO: 72), DOM7h-14-65 (SEQ ID NO: 73), DOM7h-14-74 (SEQ ID NO: 74), DOM7h-14-76 (SEQ ID NO: 75), DOM7h-14-82 (SEQ ID NO: 76), DOM7h-14-100 (SEQ ID NO: 77), DOM7h-14-101 (SEQ ID NO: 78), DOM7h-14-109 (SEQ ID NO: 79), DOM7h-14-115 (SEQ ID NO: 80), DOM7h-14-116 (SEQ ID NO: 81), DOM7h-14-119 (SEQ ID NO: 82), DOM7h-14-120 (SEQ ID NO: 83), DOM7h-14-121 (SEQ ID NO: 84), DOM7h-14-122 (SEQ ID NO: 85) and DOM7h-14-123 (SEQ ID NO: 86). In one embodiment a variant single variable domain is provided which is identical to said selected domain with the exception of one, two, three, four or five amino acid differences.

Embodiments of any aspect of the invention provide DOM7h-14 variants of good anti-serum albumin affinities. The choice of variant can allow for tailoring of half-life according to the desired therapeutic and/or prophylactic setting. For example, in one embodiment, the affinity of the variant for serum albumin is relatively high, such that the variant would be useful for inclusion in products that find utility in treating and/or preventing chronic or persistent diseases, conditions, toxicity or other chronic indications. In one embodiment, the affinity of the variant for serum albumin is relatively modest, such that the variant would be useful for inclusion in products that find utility in treating and/or preventing acute diseases, conditions, toxicity or other acute indications. In one embodiment, the affinity of the variant for serum albumin is intermediate, such that the variant would be useful for inclusion in products that find utility in treating and/or preventing acute or chronic diseases, conditions, toxicity or other acute or chronic indications.

It is conceivable that a molecule with an appropriately high affinity and specificity for serum albumin would stay in circulation long enough to have the desired therapeutic effect (Tomlinson, Nature Biotechnology 22, 521-522 (2004)). Here, a high affinity anti-SA variant would stay in serum circulation matching that of the species' serum albumin (WO2008096158). Once in circulation, any fused therapeutic agent to the AlbudAb™ variant (an AlbudAb is an anti-serum albumin dAb or immunoglobulin single variable domain), be it NCE, peptide or protein, consequently would be able to act longer on its target and exhibit a longer lasting therapeutic effect. This would allow for targeting chronic or persistent diseases without the need of frequent dosing.

A variant with moderate affinity (but specificity to SA) would only stay in serum circulation for a short time (e.g., for a few hours or a few days) allowing for the specific targeting of therapeutic targets involved in acute diseases by the fused therapeutic agent.

This way it is possible to tailor the anti-SA-containing product to the therapeutic disease area by choosing an anti-SA variant with the appropriate albumin binding affinity and/or serum half-life.

One of the properties of domain antibodies is that they can exist and bind to target in monomeric or dimeric forms. Other embodiments of any aspect of the invention provide variants which are monomeric or di- or multi-meric. A monomer dAb may be preferred for certain targets or indications where it is advantageous to prevent target cross-linking (for example, where the target is a cell surface receptor such as a receptor tyrosine kinase e.g. TNFR1). In some instances, binding as a dimer or multimer could cause receptor cross-linking of receptors on the cell surface, thus increasing the likelihood of receptor agonism and detrimental receptor signaling. Alternatively, a dAb which forms a dimer may be preferred to ensure target cross-linking or for improved binding through avidity effect, stability or solubility, for example.

For certain targeting approaches involving a multidomain construct, it may be preferable to use a monomer dAb e.g. when a dual targeting molecule is to be generated, such as a dAb-AlbudAb™ where the AlbudAb binds serum albumin, as described above, since dimerizing dAbs may lead to the formation of high molecular weight protein aggregates, for example.

An aspect of the invention provides a multispecific ligand comprising any anti-SA variant as described above and a binding moiety that specifically binds a target antigen other than SA.

An aspect of the invention provides a fusion product, e.g., a fusion protein or fusion with a peptide or NCE (new chemical entity) drug, comprising a polypeptide, protein, peptide or NCE drug fused or conjugated (for an NCE) to any variant as described above. Suitably, only a modest drop in affinity of the variant for its binding partner is observed when fused or conjugated to a partner making it useful in fusion products. In one embodiment, the invention provides a fusion protein comprising a polypeptide or peptide drug fused to a single variable domain according to the invention, optionally wherein the variant or moiety is DOM7h-14-100 (SEQ ID NO: 77).

In another embodiment, the invention provides an anti-SA single variable domain of the invention, wherein the variable domain is conjugated to a drug (optionally an NCE drug), optionally wherein the variable domain or moiety is DOM7h-14-100 (SEQ ID NO: 77).

An aspect of the invention provides a composition comprising a variant, fusion product, protein or ligand of any preceding aspect and a pharmaceutically acceptable diluent, carrier, excipient or vehicle.

An aspect of the invention provides a polypeptide fusion or conjugate comprising an anti-serum albumin dAb as disclosed herein and an incretin or insulinotropic agent, e.g., exendin-4, GLP-1(7-37), GLP-1(6-36) or any incretin or insulinotropic agent disclosed in WO06/059106, these agents being explicitly incorporated herein by reference as though written herein for inclusion in the present invention and claims below.

In another aspect, the invention provides a multispecific ligand comprising an anti-SA single variable domain of said further aspect and a binding moiety that specifically binds a target antigen other than SA.

The invention provides a nucleic acid comprising a nucleotide sequence encoding a single variable domain, a multispecific ligand or fusion protein as described in accordance with any aspect of the invention.

The invention provides a nucleic acid comprising a nucleotide sequence selected from SEQ ID NO: 87 to 101 or a nucleotide sequence that is at least 80% identical to said selected sequence. The invention provides a vector comprising the nucleic acid or an isolated host cell comprising the vector.

An aspect of the invention provides a method of treating or preventing a disease or disorder in a patient, comprising administering at least one dose of a variant, ligand, fusion product, protein or composition according to any aspect or embodiment of the invention to said patient. Another aspect provides a variant, ligand, multispecific ligand, fusion product, fusion protein, protein or composition in accordance with the invention for use as a medicament.

### BRIEF DESCRIPTION OF THE DRAWINGS

FIG. 1: Amino-acid sequence alignment for DOM7h-14 variant dAbs described in PCT/EP2010/052007. A “.” at a particular position indicates the same amino as found in DOM7h-14 at that position. The CDRs are indicated by underlining and bold text (the first underlined sequence is CDR1, the second underlined sequence is CDR2 and the third underlined sequence is CDR3).

FIG. 2: Kinetic parameters of DOM7h-14 variants. KD units=nM; Kd units=sec⁻¹; Ka units=M⁻¹ sec⁻¹. The notation A e-B means A×10^{-B} and C e D means C×10^{D}. The overall kinetic ranges in various species, as supported by the examples below, are indicated. Optional ranges are also provided for use in particular therapeutic settings (acute or chronic indications, conditions or diseases and “intermediate” for use in both chronic and acute settings). High affinity dAbs and products comprising these are useful for chronic settings. Medium affinity dAbs and products comprising these are useful for intermediate settings. Low affinity dAbs and products comprising these are useful for acute settings. The affinity in this respect is the affinity for serum albumin. Various example anti-serum dAbs and fusion proteins are listed, and these support the ranges disclosed. Many of the examples have favourable kinetics in human and one or more non-human animals (e.g., in human and Cynomolgus monkey and/or mouse). Choice of dAb or product comprising this can be tailored, according to the invention, depending on the setting (e.g., chronic or acute) to be treated therapeutically.

FIG. 3: Amino-acid sequence alignment for DOM7h-14-10 variant dAbs described herein.

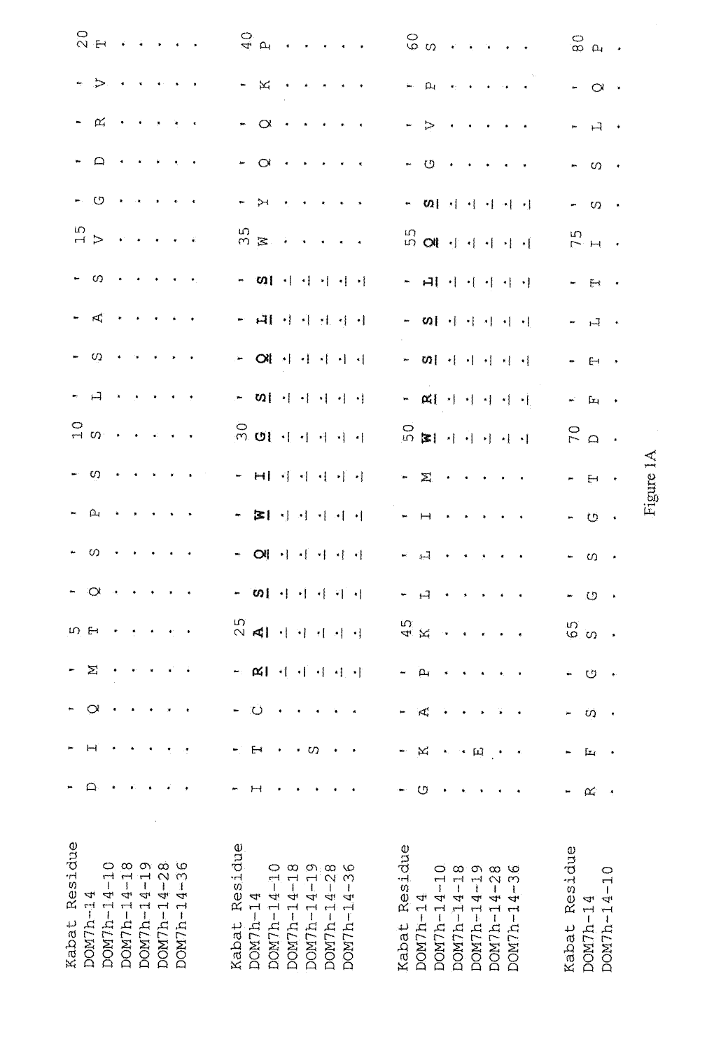

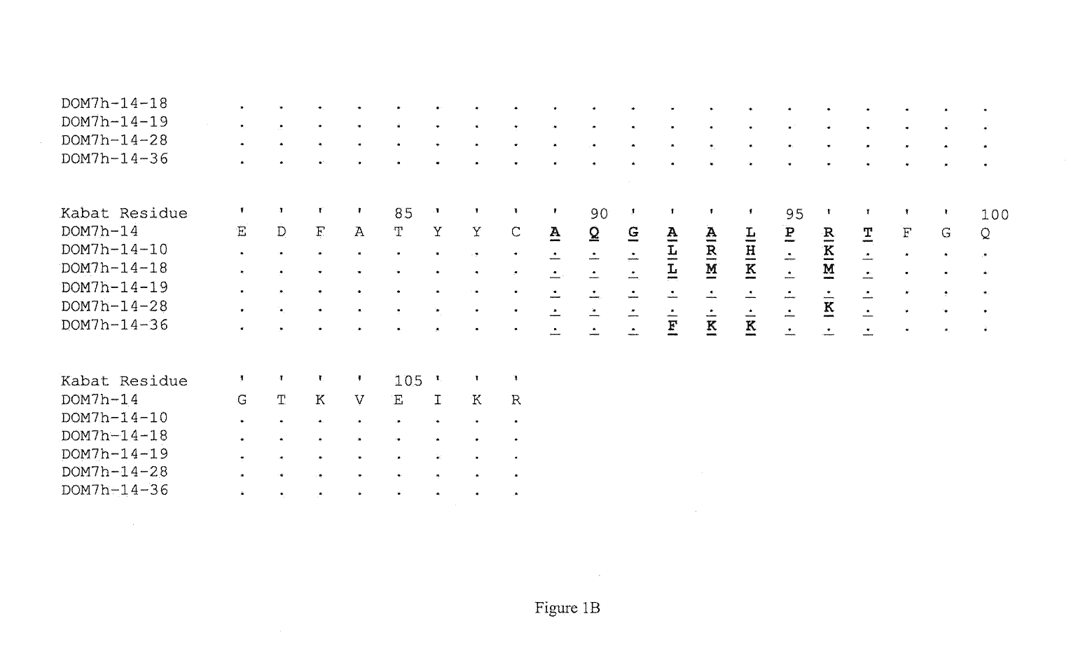

### DETAILED DESCRIPTION OF THE INVENTION

Within this specification the invention has been described, with reference to embodiments, in a way which enables a clear and concise specification to be written. It is intended and should be appreciated that embodiments may be variously combined or separated without parting from the invention.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

A “patient” is any animal, e.g., a mammal, e.g., a non-human primate (such as a baboon, rhesus monkey or Cynomolgus monkey), mouse, human, rabbit, rat, dog, cat or pig. In one embodiment, the patient is a human.

As used herein an antibody refers to IgG, IgM, IgA, IgD or IgE or a fragment (such as a Fab, Fab′, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

As used herein, “antibody format” refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a single antibody variable domain (e.g., a dAb, V_{H}, V_{HH}, V_{L}), and modified versions of any of the foregoing (e.g., modified by the covalent attachment of polyethylene glycol or other suitable polymer or a humanized V_{HH}).

The phrase “immunoglobulin single variable domain” refers to an antibody variable domain (V_{H}, V_{HH}, V_{L}) that specifically binds an antigen or epitope independently of different V regions or domains. An immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” as the term is used herein. A “single immunoglobulin variable domain” is the same as an “immunoglobulin single variable domain” as the term is used herein. A “single antibody variable domain” or an “antibody single variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized.

A “domain” is a folded protein structure which has tertiary structure independent of the rest of the protein. Generally, domains are responsible for discrete functional properties of proteins and, in many cases, may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

In the instant application, the term “prevention” and “preventing” involves administration of the protective composition prior to the induction of the disease or condition. “Treatment” and “treating” involves administration of the protective composition after disease or condition symptoms become manifest. “Suppression” or “suppressing” refers to administration of the composition after an inductive event, but prior to the clinical appearance of the disease or condition.

As used herein, the term “dose” refers to the quantity of ligand administered to a subject all at one time (unit dose), or in two or more administrations over a defined time interval. For example, dose can refer to the quantity of ligand (e.g., ligand comprising an immunoglobulin single variable domain that binds target antigen) administered to a subject over the course of one day (24 hours) (daily dose), two days, one week, two weeks, three weeks or one or more months (e.g., by a single administration, or by two or more administrations). The interval between doses can be any desired amount of time. The term “pharmaceutically effective” when referring to a dose means a sufficient amount of the ligand, domain or pharmaceutically active agent to provide the desired effect. The amount that is “effective” will vary from subject to subject, depending on the age and general condition of the individual, the particular drug or pharmaceutically active agent and the like. Thus, it is not always possible to specify an exact “effective” amount applicable for all patients. However, an appropriate “effective” dose in any individual case may be determined by one of ordinary skill in the art using routine experimentation.

Methods for pharmacokinetic analysis and determination of ligand (e.g., single variable domain, fusion protein or multi-specific ligand) half-life will be familiar to those skilled in the art. Details may be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC). Optionally, all pharmacokinetic parameters and values quoted herein are to be read as being values in a human. Optionally, all pharmacokinetic parameters and values quoted herein are to be read as being values in a mouse or rat or Cynomolgus monkey.

Half lives (t½ alpha and t½ beta) and AUC can be determined from a curve of serum concentration of ligand against time. The WinNonlin analysis package, e.g. version 5.1 (available from Pharsight Corp., Mountain View, Calif. 94040, USA) can be used, for example, to model the curve. When two-compartment modeling is used, in a first phase (the alpha phase) the ligand is undergoing mainly distribution in the patient, with some elimination. A second phase (beta phase) is the phase when the ligand has been distributed and the serum concentration is decreasing as the ligand is cleared from the patient. The t alpha half life is the half life of the first phase and the t beta half life is the half life of the second phase. Thus, in one embodiment, in the context of the present invention, the variable domain, fusion protein or ligand has a tα half life in the range of (or of about) 15 minutes or more. In one embodiment, the lower end of the range is (or is about) 30 minutes, 45 minutes, 1 hour, 2 hours, 3 hours, 4 hours, 5 hours, 6 hours, 7 hours, 10 hours, 11 hours or 12 hours. In addition, or alternatively, the variable domain, fusion protein or ligand according to the invention will have a tα half life in the range of up to and including 12 hours (or about 12 hours). In one embodiment, the upper end of the range is (or is about) 11, 10, 9, 8, 7, 6 or 5 hours. An example of a suitable range is (or is about) 1 to 6 hours, 2 to 5 hours or 3 to 4 hours.

In one embodiment, the present invention provides the variable domain, fusion protein or ligand according to the invention has a tβn one embodiment, the pre (or of about) 2.5 hours or more. In one embodiment, the lower end of the range is (or is about) 3 hours, 4 hours, 5 hours, 6 hours, 7 hours, 10 hours, 11 hours, or 12 hours. In addition, or alternatively, the tβ half life is (or is about) up to and including 21 or 25 days. In one embodiment, the upper end of the range is (or is about) 12 hours, 24 hours, 2 days, 3 days, 5 days, 10 days, 15 days, 19 days, 20 days, 21 days or 22 days. For example, the variable domain, fusion protein or ligand according to the invention will have a tβ half life in the range 12 to 60 hours (or about 12 to 60 hours). In a further embodiment, it will be in the range 12 to 48 hours (or about 12 to 48 hours). In a further embodiment still, it will be in the range 12 to 26 hours (or about 12 to 26 hours).

As an alternative to using two-compartment modeling, the skilled person will be familiar with the use of non-compartmental modeling, which can be used to determine terminal half-lives (in this respect, the term “terminal half-life” as used herein means a terminal half-life determined using non-compartmental modeling). The WinNonlin analysis package, e.g. version 5.1 (available from Pharsight Corp., Mountain View, Calif. 94040, USA) can be used, for example, to model the curve in this way. In this instance, in one embodiment the single variable domain, fusion protein or ligand has a terminal half life of at least (or at least about) 8 hours, 10 hours, 12 hours, 15 hours, 28 hours, 20 hours, 1 day, 2 days, 3 days, 7 days, 14 days, 15 days, 16 days, 17 days, 18 days, 19 days, 20 days, 21 days, 22 days, 23 days, 24 days or 25 days. In one embodiment, the upper end of this range is (or is about) 24 hours, 48 hours, 60 hours or 72 hours or 120 hours. For example, the terminal half-life is (or is about) from 8 hours to 60 hours, or 8 hours to 48 hours or 12 to 120 hours, e.g., in man.

In addition, or alternatively to the above criteria, the variable domain, fusion protein or ligand according to the invention has an AUC value (area under the curve) in the range of (or of about) 1 mg·min/ml or more. In one embodiment, the lower end of the range is (or is about) 5, 10, 15, 20, 30, 100, 200 or 300 mg·min/ml. In addition, or alternatively, the variable domain, fusion protein or ligand according to the invention has an AUC in the range of (or of about) up to 600 mg·min/ml. In one embodiment, the upper end of the range is (or is about) 500, 400, 300, 200, 150, 100, 75 or 50 mg·min/ml. Advantageously the variable domain, fusion protein or ligand will have an AUC in (or about in) the range selected from the group consisting of the following: 15 to 150 mg·min/ml, 15 to 100 mg·min/ml, 15 to 75 mg·min/ml, and 15 to 50 mg·min/ml.

“Surface Plasmon Resonance”: Competition assays can be used to determine if a specific antigen or epitope, such as human serum albumin, competes with another antigen or epitope, such as cynomolgus serum albumin, for binding to a serum albumin binding ligand described herein, such as a specific dAb. Similarly competition assays can be used to determine if a first ligand such as dAb, competes with a second ligand such as a dAb for binding to a target antigen or epitope. The term “competes” as used herein refers to substance, such as a molecule, compound, preferably a protein, which is able to interfere to any extent with the specific binding interaction between two or more molecules. The phrase “does not competitively inhibit” means that substance, such as a molecule, compound, preferably a protein, does not interfere to any measurable or significant extent with the specific binding interaction between two or more molecules. The specific binding interaction between two or more molecules preferably includes the specific binding interaction between a single variable domain and its cognate partner or target. The interfering or competing molecule can be another single variable domain or it can be a molecule that is structurally and/or functionally similar to a cognate partner or target.

The term “binding moiety” refers to a domain that specifically binds an antigen or epitope independently of a different epitope or antigen binding domain. A binding moiety may be a domain antibody (dAb) or may be a domain which is a derivative of a non-immunoglobulin protein scaffold, e.g., a scaffold selected from the group consisting of CTLA-4, lipocalin, SpA, an affibody, an avimer, GroEI, transferrin, GroES and fibronectin, which binds to a ligand other than the natural ligand (in the case of the present invention, the moiety binds serum albumin). See WO2008/096158, which discloses examples of protein scaffolds and methods for selecting antigen or epitope-specific binding domains from repertoires (see Examples 17 to 25). These specific disclosures of WO2008/096158 are expressly incorporated herein by reference as though explicitly written herein and for use with the present invention, and it is contemplated that any part of such disclosure can be incorporated into one or more claims herein).

In one embodiment, the variant or binding moiety according to any aspect or embodiment of the invention comprises one or more of the following kinetic characteristics:—
(a) The variant or moiety comprises a binding site that specifically binds human SA with a dissociation constant (KD) from (or from about) 0.1 to (or to about) 10000 nM, optionally from (or from about) 1 to (or to about) 6000 nM, as determined by surface plasmon resonance;
(b) The variant or moiety comprises a binding site that specifically binds human SA with an off-rate constant (K_{d}) from (or from about) 1.5×10⁻⁴ to (or to about) 0.1 sec⁻¹, optionally from (or from about) 3×10⁻⁴ to (or to about) 0.1 sec⁻¹ as determined by surface plasmon resonance;
(c) The variant or moiety comprises a binding site that specifically binds human SA with an on-rate constant (Kₐ) from (or from about) 2×10⁶ to (or to about) 1×10⁴ M⁻¹ sec⁻¹, optionally from (or from about) 1×10⁶ to (or to about) 2×10⁴ M⁻¹ sec⁻¹ as determined by surface plasmon resonance;
(d) The variant or moiety comprises a binding site that specifically binds Cynomolgus monkey SA with a dissociation constant (KD) from (or from about) 0.1 to (or to about) 10000 nM, optionally from (or from about) 1 to (or to about) 6000 nM, as determined by surface plasmon resonance;
(e) The variant or moiety of any preceding claim, wherein the variant comprises a binding site that specifically binds Cynomolgus monkey SA with an off-rate constant (K_{d}) from (or from about) 1.5×10⁻⁴ to (or to about) 0.1 sec⁻¹, optionally from (or from about) 3×10⁻⁴ to (or to about) 0.1 sec⁻¹ as determined by surface plasmon resonance;
(f) The variant or moiety of any preceding claim, wherein the variant comprises a binding site that specifically binds Cynomolgus monkey SA with an on-rate constant (Kₐ) from (or from about) 2×10⁶ to (or to about) 1×10⁴ M⁻¹ sec⁻¹, optionally from (or from about) 1×10⁶ to (or to about) 5×10³ M⁻¹ sec⁻¹ as determined by surface plasmon resonance;
(g) The variant or moiety comprises a binding site that specifically binds rat SA with a dissociation constant (KD) from (or from about) 1 to (or to about) 10000 nM, optionally from (or from about) 20 to (or to about) 6000 nM, as determined by surface plasmon resonance;
(h) The variant or moiety comprises a binding site that specifically binds rat SA with an off-rate constant (K_{d}) from (or from about) 2×10⁻³ to (or to about) 0.15 sec⁻¹, optionally from (or from about) 9×10⁻³ to (or to about) 0.14 sec⁻¹ as determined by surface plasmon resonance;
(i) The variant or moiety comprises a binding site that specifically binds rat SA with an on-rate constant (Kₐ) from (or from about) 2×10⁶ to (or to about) 1×10⁴ M⁻¹ sec⁻¹, optionally from (or from about) 1×10⁶ to (or to about) 3×10⁴ M⁻¹ sec⁻¹ as determined by surface plasmon resonance;
(j) The variant or moiety comprises a binding site that specifically binds mouse SA with a dissociation constant (KD) from (or from about) 1 to (or to about) 10000 nM as determined by surface plasmon resonance;
(k) The variant or moiety comprises a binding site that specifically binds mouse SA with an off-rate constant (K_{d}) from (or from about) 2×10⁻³ to (or to about) 0.15 sec as determined by surface plasmon resonance; and/or
(l) The variant or moiety comprises a binding site that specifically binds mouse SA with an on-rate constant (Kₐ) from (or from about) 2×10⁶ to (or to about) 1×10⁴ M⁻¹ sec⁻¹, optionally from (or from about) 2×10⁶ to (or to about) 1.5×10⁴ M⁻¹ sec⁻¹ as determined by surface plasmon resonance.

Optionally, the variant or moiety has
I: a KD according to (a) and (d), a K_{d} according to (b) and (e), and a Kₐ according to (c) and (f); or
II: a KD according to (a) and (g), a K_{d} according to (b) and (h), and a Kₐ according to (c) and (i); or
III: a KD according to (a) and (j), a K_{d} according to (b) and (k), and a Kₐ according to (c) and (I); or
IV: kinetics according to I and II; or
V: kinetics according to I and III; or
VI: kinetics according to I, II and III.

The invention also provides a ligand comprising a variant or moiety of any preceding aspect or embodiment of the invention. For example, the ligand can be a dual-specific ligand (see WO04003019 for examples of dual-specific ligands). In one aspect, the invention provides a multispecific ligand comprising an anti-SA variant or moiety of any preceding aspect or embodiment of the invention and a further binding moiety that specifically binds a target antigen other than SA. The or each binding moiety can be any binding moiety that specifically binds a target, e.g., the moiety is an antibody, antibody fragment, scFv, Fab, dAb or a binding moiety comprising a non-immunoglobulin protein scaffold. Such moieties are disclosed in detail in WO2008/096158 (see examples 17 to 25, which disclosure is incorporated herein by reference). Examples of non-immunoglobulin scaffolds are CTLA-4, lipocallin, staphylococcal protein A (spA), Affibody™, Avimers™, GroEL and fibronectin.

In one embodiment, a linker is provided between the anti-target binding moiety and the anti-SA single variant or moiety, the linker comprising the amino acid sequence AST, optionally ASTSGPS, e.g., where anti-SA and anti-target dAbs are used. Alternative linkers are described in WO2007085814 (incorporated herein by reference) and WO2008/096158 (see the passage at page 135, line 12 to page 140, line 14, which disclosure and all sequences of linkers are expressly incorporated herein by reference as though explicitly written herein and for use with the present invention, and it is contemplated that any part of such disclosure can be incorporated into one or more claims herein) and WO2009/068649.

In one embodiment of the multispecific ligand, the target antigen may be, or be part of, polypeptides, proteins or nucleic acids, which may be naturally occurring or synthetic. In this respect, the ligand of the invention may bind the target antigen and act as an antagonist or agonist (e.g., EPO receptor agonist). One skilled in the art will appreciate that the choice is large and varied. They may be for instance, human or animal proteins, cytokines or growth factors, cytokine or growth factor receptors, where cytokine receptors include receptors for cytokines, enzymes, co-factors for enzymes or DNA binding proteins. As used herein, the term “antagonist of Tumor Necrosis Factor Receptor 1 (TNFR1)” or “anti-TNFR1 antagonist” or the like refers to an agent (e.g., a molecule, a compound) which binds TNFR1 and can inhibit a (i.e., one or more) function of TNFR1. For example, an antagonist of TNFR1 can inhibit the binding of TNF alpha to TNFR1 and/or inhibit signal transduction mediated through TNFR1. Accordingly, TNFR1-mediated processes and cellular responses (e.g., TNF alpha-induced cell death in a standard L929 cytotoxicity assay) can be inhibited with an antagonist of TNFR1.

In one embodiment, the multispecific ligand comprises an anti-SA dAb variant or moiety of the invention and an anti-TNFR1 binding moiety, e.g., an anti-TNFR1 dAb. Optionally, the ligand has only one anti-TNFR1 binding moiety (e.g., dAb) to reduce the chance of receptor cross-linking. Anti-TNFR1 dAbs are described, for example, in WO2006/038027, WO2007/049017, WO2008149148 and WO2010/081787 (the amino acid sequences of which and the nucleotide sequence of which, as disclosed in those PCT applications, are expressly incorporated herein by reference as though explicitly written herein and for use with the present invention, and it is contemplated that any part of such disclosures can be incorporated into one or more claims herein).

In one embodiment, the ligand of the invention is a fusion protein comprising a variant or moiety of the invention fused directly or indirectly to one or more polypeptides. For example, the fusion protein can be a “drug fusion” as disclosed in WO2005/118642 (the disclosure of which is incorporated herein by reference), comprising a variant or moiety of the invention and a polypeptide drug as defined in that PCT application.

As used herein, “drug” refers to any compound (e.g., small organic molecule, nucleic acid, polypeptide) that can be administered to an individual to produce a beneficial, therapeutic or diagnostic effect through binding to and/or altering the function of a biological target molecule in the individual. The target molecule can be an endogenous target molecule encoded by the individual's genome (e.g. an enzyme, receptor, growth factor, cytokine encoded by the individual's genome) or an exogenous target molecule encoded by the genome of a pathogen (e.g. an enzyme encoded by the genome of a virus, bacterium, fungus, nematode or other pathogen). Suitable drugs for use in fusion proteins and conjugates comprising an anti-SA dAb variant of the invention are disclosed in WO2005/118642 and WO2006/059106 (the entire disclosures of which are incorporated herein by reference, and including the entire list of specific drugs as though this list were expressly written herein, and it is contemplated that such incorporation provides disclosure of specific drugs for inclusion in claims herein). For example, the drug can be glucagon-like peptide 1 (GLP-1) or a variant, interferon alpha 2b or a variant or exendin-4 or a variant.

In one embodiment, the invention provides a drug conjugate as defined and disclosed in WO2005/118642 and WO2006/059106, wherein the conjugate comprises a variant or moiety of the invention. In one example, the drug is covalently linked to the variant or moiety (e.g., the variant or moiety and the drug are expressed as part of a single polypeptide). Alternatively, in an example, the drug is non-covalently bonded or associated with the variant or moiety. The drug can be covalently or noncovalently bonded to the variant or moiety directly or indirectly (e.g., through a suitable linker and/or noncovalent binding of complementary binding partners (e.g., biotin and avidin)). When complementary binding partners are employed, one of the binding partners can be covalently bonded to the drug directly or through a suitable linker moiety, and the complementary binding partner can be covalently bonded to the variant or moiety directly or through a suitable linker moiety. When the drug is a polypeptide or peptide, the drug composition can be a fusion protein, wherein the polypeptide or peptide, drug and the polypeptide binding moiety are discrete parts (moieties) of a continuous polypeptide chain. As described herein, the polypeptide binding moieties and polypeptide drug moieties can be directly bonded to each other through a peptide bond, or linked through a suitable amino acid, or peptide or polypeptide linker.

A ligand which contains one single variable domain (monomer) variant or moiety of the invention or more than one single variable domain or moiety (multimer, fusion protein, conjugate, and dual specific ligand as defined herein) which specifically binds to serum albumin, can further comprise one or more entities selected from, but preferably not limited to a label, a tag, an additional single variable domain, a dAb, an antibody, an antibody fragment, a marker and a drug. One or more of these entities can be located at either the COOH terminus or at the N terminus or at both the N terminus and the COOH terminus of the ligand comprising the single variable domain or moiety, (either immunoglobulin or non-immunoglobulin single variable domain). One or more of these entities can be located at either the COOH terminus, or the N terminus, or both the N terminus and the COOH terminus of the single variable domain or moiety which specifically binds serum albumin of the ligand which contains one single variable domain (monomer) or moiety or more than one single variable domains or moieties (multimer, fusion protein, conjugate, and dual specific ligand as defined herein). Non-limiting examples of tags which can be positioned at one or both of these termini include a HA, his or a myc tag. The entities, including one or more tags, labels and drugs, can be bound to the ligand which contains one single variable domain (monomer) or more than one single variable domain or moiety (multimer, fusion protein, conjugate, and dual specific ligand as defined herein), which binds serum albumin, either directly or through linkers as described above.

Also encompassed herein is an isolated nucleic acid encoding any of the variants, moieties, fusion proteins, conjugates or ligands described herein, e.g., a ligand which contains one single variable domain (monomer) variant of the invention or more than one single variable domain (e.g., multimer, fusion protein, conjugate, and dual specific ligand as defined herein) variant which specifically binds to serum albumin, or which specifically binds both human serum albumin and at least one non-human serum albumin, or functionally active fragments thereof. Also encompassed herein is a vector and/or an expression vector, a host cell comprising the vector, e.g., a plant or animal cell and/or cell line transformed with a vector, a method of expressing and/or producing one or more variants, moieties, fusion proteins or ligands which contains one single variable domain (monomer) variant or moiety or more than one single variable domain variants or moieties (e.g., multimer, fusion protein, conjugate, and dual specific ligand as defined herein) which specifically binds to serum albumin, or fragment(s) thereof encoded by said vectors, including in some instances culturing the host cell so that the one or more variants, moieties, fusion proteins or ligands or fragments thereof are expressed and optionally recovering the ligand which contains one single variable domain or moiety (monomer) or more than one single variable domain or moiety (e.g., multimer, fusion protein, conjugate, and dual specific ligand as defined herein) which specifically binds to serum albumin, from the host cell culture medium. Also encompassed are methods of contacting a ligand described herein with serum albumin, including serum albumin and/or non-human serum albumin(s), and/or one or more targets other than serum albumin, where the targets include biologically active molecules, and include animal proteins, cytokines as listed above, and include methods where the contacting is in vitro as well as administering any of the variants, moieties, fusion proteins or ligands described herein to an individual host animal or cell in vivo and/or ex vivo. Preferably, administering ligands described herein which comprises a single variable domain (immunoglobulin or non-immunoglobulin) directed to serum albumin and/or non-human serum albumin(s), and one or more domains directed to one or more targets other than serum albumin, will increase the half life, including the T beta and/or terminal half life, of the anti-target ligand. Nucleic acid molecules encoding the variants, fusion proteins or single domain containing ligands or fragments thereof, including functional fragments thereof, are contemplated herein. Vectors encoding the nucleic acid molecules, including but preferably not limited to expression vectors, are contemplated herein, as are host cells from a cell line or organism containing one or more of these expression vectors. Also contemplated are methods of producing any variant, fusion protein or ligand, including, but preferably not limited to any of the aforementioned nucleic acids, vectors and host cells.

An aspect of the invention provides a nucleic acid comprising a nucleotide sequence encoding a variant according to the invention or a multispecific ligand of the invention or fusion protein of the invention.

or a nucleotide sequence that is at least 70, 75, 80, 85, 90, 95, 96, 97, 98 or 99% identical to said selected sequence.

An aspect of the invention provides a vector comprising the nucleic acid of the invention. An aspect of the invention provides an isolated host cell comprising the vector.

Reference is made to WO2008/096158 for details of library vector systems, combining single variable domains, characterization of dual specific ligands, structure of dual specific ligands, scaffolds for use in constructing dual specific ligands, uses of anti-serum albumin dAbs and multispecific ligands and half-life-enhanced ligands, and compositions and formulations of comprising anti-serum albumin dAbs. These disclosures are incorporated herein by reference to provide guidance for use with the present invention, including for variants, moieties, ligands, fusion proteins, conjugates, nucleic acids, vectors, hosts and compositions of the present invention.

### SEQUENCES

TABLE 1
Amino Acid Sequences of DOM7h-14 Variant dAbs

DOM7h-14-10
(SEQ ID NO: 1)
DIQMTQSPSSLSASVGDRVTITCRASQWIGSQLSWYQQKPGKAPKLL
IMWRSSLQSGVPSRFSGSGSGTDFTLTISSLQPEDFATYYCAQGLRH
PKTFGQGTKVEIKR
DOM7h-14-18
(SEQ ID NO: 2)
DIQMTQSPSSLSASVGDRVTITCRASQWIGSQLSWYQQKPGKAPKLL
IMWRSSLQSGVPSRFSGSGSGTDFTLTISSLQPEDFATYYCAQGLMK
PMTFGQGTKVEIKR
DOM7h-14-19
(SEQ ID NO: 3)
DIQMTQSPSSLSASVGDRVTISCRASQWIGSQLSWYQQKPGEAPKLL
IMWRSSLQSGVPSRFSGSGSGTDFTLTISSLQPEDFATYYCAQGAAL
PRTFGQGTKVEIKR
DOM7h-14-28
(SEQ ID NO: 4)
DIQMTQSPSSLSASVGDRVTITCRASQWIGSQLSWYQQKPGKAPKLL
IMWRSSLQSGVPSRFSGSGSGTDFTLTISSLQPEDFATYYCAQGAAL
PKTFGQGTKVEIKR
DOM7h-14-36
(SEQ ID NO: 5)
DIQMTQSPSSLSASVGDRVTITCRASQWIGSQLSWYQQKPGKAPKLL
IMWRSSLQSGVPSRFSGSGSGTDFTLTISSLQPEDFATYYCAQGFKK
PRTFGQGTKVEIKR

TABLE 2
Nucleotide Sequences of DOM7h-14 Variant dAbs

DOM7h-14-10
(SEQ ID NO: 6)
GACATCCAGA TGACCCAGTC TCCATCCTCC CTGTCTGCAT
CTGTAGGAGA CCGTGTCACC ATCACTTGCC GGGCAAGTCA
GTGGATTGGG TCTCAGTTAT CTTGGTACCA GCAGAAACCA
GGGAAAGCCC CTAAGCTCCT GATCATGTGG CGTTCCTCGT
TGCAAAGTGG GGTCCCATCA CGTTTCAGTG GCAGTGGATC
TGGGACAGAT TTCACTCTCA CCATCAGCAG TCTGCAACCT
GAAGATTTTG CTACGTACTA CTGTGCTCAG GGTTTGAGGC
ATCCTAAGAC GTTCGGCCAA GGGACCAAGG TGGAAATCAA
ACGG
DOM7h-14-18
(SEQ ID NO: 7)
GACATCCAGA TGACCCAGTC TCCATCCTCC CTGTCTGCAT
CTGTAGGAGA CCGTGTCACC ATCACTTGCC GGGCAAGTCA
GTGGATTGGG TCTCAGTTAT CTTGGTACCA GCAGAAACCA
GGGAAAGCCC CTAAGCTCCT GATCATGTGG CGTTCCTCGT
TGCAAAGTGG GGTCCCATCA CGTTTCAGTG GCAGTGGATC
TGGGACAGAT TTCACTCTCA CCATCAGCAG TCTGCAACCT
GAAGATTTTG CTACGTACTA CTGTGCTCAG GGTCTTATGA
AGCCTATGAC GTTCGGCCAA GGGACCAAGG TGGAAATCAA
ACGG
DOM7h-14-19
(SEQ ID NO: 8)
GACATCCAGA TGACCCAGTC TCCATCCTCC CTGTCTGCAT
CTGTAGGAGA CCGTGTCACC ATCTCTTGCC GGGCAAGTCA
GTGGATTGGG TCTCAGTTAT CTTGGTACCA GCAGAAACCA
GGGGAAGCCC CTAAGCTCCT GATCATGTGG CGTTCCTCGT
TGCAAAGTGG GGTCCCATCA CGTTTCAGTG GCAGTGGATC
TGGGACAGAT TTCACTCTCA CCATCAGCAG TCTGCAACCT
GAAGATTTTG CTACGTACTA CTGTGCTCAG GGTGCGGCGT
TGCCTAGGAC GTTCGGCCAA GGGACCAAGG TGGAAATCAA
ACGG
DOM7h-14-28
(SEQ ID NO: 9)
GACATCCAGA TGACCCAGTC TCCATCCTCC CTGTCTGCAT
CTGTAGGAGA CCGTGTCACC ATCACTTGCC GGGCAAGTCA
GTGGATTGGG TCTCAGTTAT CTTGGTACCA GCAGAAACCA
GGGAAAGCCC CTAAGCTCCT GATCATGTGG CGTTCCTCGT
TGCAAAGTGG GGTCCCATCA CGTTTCAGTG GCAGTGGATC
TGGGACAGAT TTCACTCTCA CCATCAGCAG TCTGCAACCT
GAAGATTTTG CTACATACTA CTGTGCTCAG GGTGCGGCGT
TGCCTAAGAC GTTCGGCCAA GGGACCAAGG TGGAAATCAA
ACGG
DOM7h-14-36
(SEQ ID NO: 10)
GACATCCAGA TGACCCAGTC TCCATCCTCC CTGTCTGCAT
CTGTAGGAGA CCGTGTCACC ATCACTTGCC GGGCAAGTCA
GTGGATTGGG TCTCAGTTAT CTTGGTACCA GCAGAAACCA
GGGAAAGCCC CTAAGCTCCT GATCATGTGG CGTTCCTCGT
TGCAAAGTGG GGTCCCATCA CGTTTCAGTG GCAGTGGATC
TGGGACAGAT TTCACTCTCA CCATCAGCAG TCTGCAACCT
GAAGATTTTG CTACGTACTA CTGTGCTCAG GGTTTTAAGA
AGCCTCGGAC GTTCGGCCAA GGGACCAAGG TGGAAATCAA
ACGG

TABLE 3
Anti-serum albumin dAb(DOM7h) fusions

(used in Rat studies):-
DOM7h-14/Exendin-4 fusion DMS number 7138
Amino acid sequence
(SEQ ID NO: 11)
HGEGTFTSDLSKQMEEEAVRLFIEWLKNGGPSSGAPPPSGGGGGSGGGG
SGGGGSDIQMTQSPSSLSASVGDRVTITCRASQWIGSQLSWYQQKPGKA
PKLLIMWRSSLQSGVPSRFSGSGSGTDFTLTISSLQPEDFATYYCAQGA
ALPRTFGQGTKVEIKR
Nucleotide sequence
(SEQ ID NO: 12)
CATGGTGAAGGAACATTTACCAGTGACTTGTCAAAACAGATGGAAGAGG
AGGCAGTGCGGTTATTTATTGAGTGGCTTAAGAACGGAGGACCAAGTAG
CGGGGCACCTCCGCCATCGGGTGGTGGAGGCGGTTCAGGCGGAGGTGGC
AGCGGCGGTGGCGGGTCGGACATCCAGATGACCCAGTCTCCATCCTCCC
TGTCTGCATCTGTAGGAGACCGTGTCACCATCACTTGCCGGGCAAGTCA
GTGGATTGGGTCTCAGTTATCTTGGTACCAGCAGAAACCAGGGAAAGCC
CCTAAGCTCCTGATCATGTGGCGTTCCTCGTTGCAAAGTGGGGTCCCAT
CACGTTTCAGTGGCAGTGGATCTGGGACAGATTTCACTCTCACCATCAG
CAGTCTGCAACCTGAAGATTTTGCTACGTACTACTGTGCTCAGGGTGCG
GCGTTGCCTAGGACGTTCGGCCAAGGGACCAAGGTGGAAATCAAACGG
DOM7h-14-10/Exendin-4 fusion DMS number 7139
Amino acid sequence
(SEQ ID NO: 13)
HGEGTFTSDLSKQMEEEAVRLFIEWLKNGGPSSGAPPPSGGGGGSGGGG
SGGGGSDIQMTQSPSSLSASVGDRVTITCRASQWIGSQLSWYQQKPGKA
PKLLIMWRSSLQSGVPSRFSGSGSGTDFTLTISSLQPEDFATYYCAQGL
RHPKTFGQGTKVEIKR
Nucleotide sequence
(SEQ ID NO: 14)
CATGGTGAAGGAACATTTACCAGTGACTTGTCAAAACAGATGGAAGAGG
AGGCAGTGCGGTTATTTATTGAGTGGCTTAAGAACGGAGGACCAAGTAG
CGGGGCACCTCCGCCATCGGGTGGTGGAGGCGGTTCAGGCGGAGGTGGC
AGCGGCGGTGGCGGGTCGGACATCCAGATGACCCAGTCTCCATCCTCCC
TGTCTGCATCTGTAGGAGACCGTGTCACCATCACTTGCCGGGCAAGTCA
GTGGATTGGGTCTCAGTTATCTTGGTACCAGCAGAAACCAGGGAAAGCC
CCTAAGCTCCTGATCATGTGGCGTTCCTCGTTGCAAAGTGGGGTCCCAT
CACGTTTCAGTGGCAGTGGATCTGGGACAGATTTCACTCTCACCATCAG
CAGTCTGCAACCTGAAGATTTTGCTACGTACTACTGTGCTCAGGGTTTG
AGGCATCCTAAGACGTTCGGCCAAGGGACCAAGGTGGAAATCAAACGG
DOM7h-14-18/Exendin-4 fusion DMS number 7140
Amino acid sequence
(SEQ ID NO: 15)
HGEGTFTSDLSKQMEEEAVRLFIEWLKNGGPSSGAPPPSGGGGGSGGGG
SGGGGSDIQMTQSPSSLSASVGDRVTITCRASQWIGSQLSWYQQKPGKA
PKLLIMWRSSLQSGVPSRFSGSGSGTDFTLTISSLQPEDFATYYCAQGL
MKPMTFGQGTKVEIKR
Nucleotide sequence
(SEQ ID NO: 16)
CATGGTGAAGGAACATTTACCAGTGACTTGTCAAAACAGATGGAAGAGG
AGGCAGTGCGGTTATTTATTGAGTGGCTTAAGAACGGAGGACCAAGTAG
CGGGGCACCTCCGCCATCGGGTGGTGGAGGCGGTTCAGGCGGAGGTGGC
AGCGGCGGTGGCGGGTCGGACATCCAGATGACCCAGTCTCCATCCTCCC
TGTCTGCATCTGTAGGAGACCGTGTCACCATCACTTGCCGGGCAAGTCA
GTGGATTGGGTCTCAGTTATCTTGGTACCAGCAGAAACCAGGGAAAGCC
CCTAAGCTCCTGATCATGTGGCGTTCCTCGTTGCAAAGTGGGGTCCCAT
CACGTTTCAGTGGCAGTGGATCTGGGACAGATTTCACTCTCACCATCAG
CAGTCTGCAACCTGAAGATTTTGCTACGTACTACTGTGCTCAGGGTCTT
ATGAAGCCTATGACGTTCGGCCAAGGGACCAAGGTGGAAATCAAACGG
DOM7h-14-19/Exendin-4 fusion DMS number 7141
Amino acid sequence
(SEQ ID NO: 17)
HGEGTFTSDLSKQMEEEAVRLFIEWLKNGGPSSGAPPPSGGGGGSGGGG
SGGGGSDIQMTQSPSSLSASVGDRVTISCRASQWIGSQLSWYQQKPGEA
PKLLIMWRSSLQSGVPSRFSGSGSGTDFTLTISSLQPEDFATYYCAQGA
ALPRTFGQGTKVEIKR
Nucleotide sequence
(SEQ ID NO: 18)
CATGGTGAAGGAACATTTACCAGTGACTTGTCAAAACAGATGGAAGAGG
AGGCAGTGCGGTTATTTATTGAGTGGCTTAAGAACGGAGGACCAAGTAG
CGGGGCACCTCCGCCATCGGGTGGTGGAGGCGGTTCAGGCGGAGGTGGC
AGCGGCGGTGGCGGGTCGGACATCCAGATGACCCAGTCTCCATCCTCCC
TGTCTGCATCTGTAGGAGACCGTGTCACCATCTCTTGCCGGGCAAGTCA
GTGGATTGGGTCTCAGTTATCTTGGTACCAGCAGAAACCAGGGGAAGCC
CCTAAGCTCCTGATCATGTGGCGTTCCTCGTTGCAAAGTGGGGTCCCAT
CACGTTTCAGTGGCAGTGGATCTGGGACAGATTTCACTCTCACCATCAG
CAGTCTGCAACCTGAAGATTTTGCTACGTACTACTGTGCTCAGGGTGCG
GCGTTGCCTAGGACGTTCGGCCAAGGGACCAAGGTGGAAATCAAACGG
DOM7h14-10/G4SC-NCE fusion
Amino acid sequence encoding DOM7h14-10/G4SC
(SEQ ID NO: 19)
DIQMTQSPSSLSASVGDRVTITCRASQWIGSQLSWYQQKPGKAPKLLIM
WRSSLQSGVPSRFSGSGSGTDFTLTISSLQPEDFATYYCAQGLRHPKTF
GQGTKVEIKRGGGGSC
The C-terminal cysteine can be linked to a new
chemical entity (pharmaceutical chemical
compound, NCE), eg using maleimide linkage.
Nucleotide sequence encoding DOM7h14-10/G4SC
(SEQ ID NO: 20)
GACATCCAGATGACCCAGTCTCCATCCTCCCTGTCTGCATCTGTAGGAG
ACCGTGTCACCATCACTTGCCGGGCAAGTCAGTGGATTGGGTCTCAGTT
ATCTTGGTACCAGCAGAAACCAGGGAAAGCCCCTAAGCTCCTGATCATG
TGGCGTTCCTCGTTGCAAAGTGGGGTCCCATCACGTTTCAGTGGCAGTG
GATCTGGGACAGATTTCACTCTCACCATCAGCAGTCTGCAACCTGAAGA
TTTTGCTACGTACTACTGTGCTCAGGGTTTGAGGCATCCTAAGACGTTC
GGCCAAGGGACCAAGGTGGAAATCAAACGGGGTGGCGGAGGGGGTTCCT
GT
DOM7h14-10/TVAAPSC fusion
Amino acid sequence
(SEQ ID NO: 21)
DIQMTQSPSSLSASVGDRVTITCRASQWIGSQLSWYQQKPGKAPKLLIM
WRSSLQSGVPSRFSGSGSGTDFTLTISSLQPEDFATYYCAQGLRHPKTF
GQGTKVEIKRTVAAPSC
The C-terminal cysteine can be linked to a new
chemical entity (pharmaceutical chemical
compound, NCE), eg using maleimide linkage.
Nucleotide sequence
(SEQ ID NO: 22)
GACATCCAGATGACCCAGTCTCCATCCTCCCTGTCTGCATCTGTAGGAG
ACCGTGTCACCATCACTTGCCGGGCAAGTCAGTGGATTGGGTCTCAGTT
ATCTTGGTACCAGCAGAAACCAGGGAAAGCCCCTAAGCTCCTGATCATG
TGGCGTTCCTCGTTGCAAAGTGGGGTCCCATCACGTTTCAGTGGCAGTG
GATCTGGGACAGATTTCACTCTCACCATCAGCAGTCTGCAACCTGAAGA
TTTTGCTACGTACTACTGTGCTCAGGGTTTGAGGCATCCTAAGACGTTC
GGCCAAGGGACCAAGGTGGAAATCAAACGGACCGTCGCTGCTCCATCTT
GT

Where a myc-tagged molecule is indicated in this table, this was the version used in PK studies in the examples. Where no myc-tagged sequences are given, the PK studies in the examples were not done with myc-tagged material, i.e., the studies were done with the non-tagged constructs shown.

### EXEMPLIFICATION

All numbering in the experimental section is according to Kabat (Kabat, E. A. National Institutes of Health (US) & Columbia University. Sequences of proteins of immunological interest, edn 5 (US Dept. Of Health and Human Services Public Health Service, National Institutes of Health, Bethesda, Md., 1991)).

### Example 1

### Vk Affinity Maturation

### Selections:

HSA (Human Serum Albumin) and RSA (Rat Serum Albumin) antigens were obtained from Sigma (essentially fatty acid free, ˜99% (agarose gel electrophoresis), lyophilized powder Cat. No. A3782 and A6414 respectively)

Biotinylated products of above two antigens were made by using EZ Link Sulfo-NHS-SS-Biotin (Pierce, Cat. No. 21331). Free biotin reagent was removed by passing the samples twice through PD10 desalting column followed by overnight dialysis against 1000× excess volume of PBS at 4° C. Resulting product was tested by mass spec and 1-2 biotins per molecule were observed.

### Affinity Maturation Libraries:

Both error-prone and CDR libraries were created using DOM7h-14 parental dAbs (see WO2008/096158 for the sequences of DOM7h-14). The CDR libraries were generated in the pDOM4 vector and the error prone libraries were generated in the pDOM33 vector (to allow for selection with or without protease treatment). Vector pDOM4, is a derivative of the Fd phage vector in which the gene III signal peptide sequence is replaced with the yeast glycolipid anchored surface protein (GAS) signal peptide. It also contains a c-myc tag between the leader sequence and gene III, which puts the gene III back in frame. This leader sequence functions well both in phage display vectors but also in other prokaryotic expression vectors and can be universally used. pDOM33 is a modified version of the pDOM4 vector where the c-myc tag has been removed which renders the dAb-phage fusion resistant to the protease trypsin. This allows the use of trypsin within the phage selection to select for dAbs that are more protease stable (see WO2008149143).

For error-prone maturation libraries, plasmid DNA encoding the dAb to be matured was amplified by PCR, using the GENEMORPH® II RANDOM MUTAGENESIS KIT (random, unique mutagenesis kit, Stratagene). The product was digested with Sal I and Not I and used in a ligation reaction with cut phage vector pDOM33.

For the CDR libraries, PCR reactions were performed using degenerate oligonucleotides containing NNK or NNS codons to diversify the required positions in the dAb to be affinity matured. Assembly PCR was then used to generate a full length diversified insert. The insert was digested with Sal I and Not I and used in a ligation reaction with pDOM4 for mutagenesis of multiple residues and pDOM5 for mutagenesis of single residues. The pDOM5 vector is a pUC119-based expression vector where protein expression is driven by the LacZ promoter. A GAS1 leader sequence (see WO 2005/093074) ensures secretion of isolated, soluble dAbs into the periplasm and culture supernatant of E. coli. dAbs are cloned SalI/NotI in this vector, which appends a myc tag at the C-terminus of the dAb. This protocol using SalI and Not I results in inclusion of an ST amino acid sequence at the N-terminus.

The ligation produced by either method was then used to transform E. coli strain TB1 by electroporation and the transformed cells plated on 2×TY agar containing 15 μg/ml tetracycline, yielding library sizes of >5×10⁷ clones.

The error-prone libraries had the following average mutation rate and size: DOM7h-14 (2.9 mutations per dAb), size:5.4×10⁸.

Each CDR library has four amino acid diversity. Two libraries were generated for each of CDRs 1 and 3, and one library for CDR2. The positions diversified within each library are as follows (amino acids based on VK dummy DPK9 sequence):

| | Library size |
|---|---|
| | DOM7h-14 |
| 1 - Q27, S28, S30, S31 (CDR1) | 5.8 × 10⁷ |
| 2 - S30, S31, Y32, N34 (CDR1) | 4.2 × 10⁸ |
| 3 - Y49, A50, A51, S53 (CDR2) | 2.4 × 10⁸ |
| 4 - Q89, S91, Y92, S93 (CDR3) | 2.5 × 10⁸ |
| 5 - Y92, Y93, T94, N96 (CDR3) | 3.3 × 10⁸ |

### Example 2

### Selection Strategies

Three phage selection strategies were adopted for Vκ AlbudAb™ (anti-serum albumin dAb) affinity maturation:

1) Selections against HSA only:
Three rounds of selection against HSA were carried out. The error prone libraries and each CDR library were selected as an individual pool in all rounds. The first round of selection was performed against HSA passively coated onto an immunotube at 1 mg/ml. Round 2 was performed against 100 nM HSA and round 3 against 10 nM (CDR selections) or 20 or 100 nM (Error prone selections) HSA, both as soluble selections followed by a fourth round of selection with the error prone libraries against 1.5 nM HSA as a soluble selection. The error prone libraries were eluted with 0.1M glycine pH 2.0 before neutralisation with 1M Tris pH 8.0 and the CDR libraries were eluted with 1 mg/ml trypsin before infection into log phase TG1 cells. The third round of each selection was subcloned into pDOM5 for screening. Soluble selections used biotinylated HSA.

2) Trypsin Selections Against HSA:
In order to select dAbs with increased protease resistance compared to the parental clone and with potentially improved biophysical properties, trypsin was used in phage selections (see WO2008149143). Four rounds of selection were preformed against HSA. The first round of selection of error prone libraries was performed against passively coated HSA at 1 mg/ml without trypsin; the second round against passively coated HSA at 1 mg/ml with 20 μg/ml trypsin for 1 hour at 37° C.; the third round selection was performed by soluble selection using biotinylated HSA against 100 nM HSA with 20 μg/ml or 100 μg/ml trypsin for 1 hour at 37° C. The final round of selection was performed by soluble selection using biotinylated HSA against 100 nM HSA with 100 μg/ml trypsin overnight at 37° C.

3) Cross-Over Selections Against HSA (Round 1) and RSA (Rounds 2-4):
The first round selection was carried out against 1 mg/ml passively coated HSA or 1 μM HSA (soluble selection), followed by a further three rounds of soluble selections against biotinylated RSA at concentrations of 1 μM for round 1, 100 nm for round 2 and 20 nM, 10 nM or 1 nM for round 3.

### Screening Strategy and Affinity Determination:

In each case after selection a pool of phage DNA from the appropriate round of selection is prepared using a QIAfilter midiprep kit (Qiagen), the DNA is digested using the restriction enzymes Sal1 and Not1 and the enriched V genes are ligated into the corresponding sites in pDOM5 the soluble expression vector which expresses the dAb with a myc tag (see PCT/EP2008/067789). The ligated DNA is used to electro-transform E. coli HB 2151 cells which are then grown overnight on agar plates containing the antibiotic carbenicillin. The resulting colonies are individually assessed for antigen binding. In each case at least 96 clones were tested for binding to HSA, CSA (Cynomolgus monkey Serum Albumin), MSA (mouse serum albumin) and RSA by BIAcore™ (surface plasmon resonance). MSA antigen was obtained from Sigma (essentially fatty acid free, ˜99% (agarose gel electrophoresis), lyophilized powder Cat. No. A3559) and CSA was purified from Cynomolgus serum albumin using prometic blue resin (Amersham). Soluble dAb fragments were produced in bacterial culture in ONEX culture media (Novagen) overnight at 37° C. in 96 well plates. The culture supernatant containing soluble dAb was centrifuged and analysed by BIAcore for binding to high density HSA, CSA, MSA and RSA CM5 chips. Clones were found to bind to all these species of serum albumin by off-rate screening. The clones were sequenced revealing unique dAb sequences.

The minimum identity to parent (at the amino acid level) of the clones selected was 96.3% (DOM7h-14-10: 96.3%, DOM7h-14-18: 96.3%, DOM7h-14-19: 98.2%, DOM7h-14-28: 99.1%, DOM7h-14-36: 97.2%)

Unique dAbs were expressed as bacterial supernatants in 2.5 L shake flasks in Onex media at 30° C. for 48 hrs at 250 rpm. dAbs were purified from the culture media by absorption to protein L agarose followed by elution with 10 mM glycine pH2.0. Binding to HSA, CSA, MSA and RSA by BIAcore was confirmed using purified protein at 3 concentrations 1 μM, 500 nM and 50 nM. To determine the binding affinity (K_{D}) of the AlbudAbs to each serum albumin; purified dAbs were analysed by BIAcore over albumin concentration range from 5000 nM to 39 nM (5000 nM, 2500 nM, 1250 nM, 625 nM, 312 nM, 156 nM, 78 nM, 39 nM).

TABLE 4

| | Affinity (K_{D}) | | |
|---|---|---|---|
| AlbudAb | to SA (nM) | Kd | Ka |
| | Rat | | |
| DOM7h-14 | 60 | 2.095E−01 | 4.00E+06 |
| DOM7h-14-10 | 4 | 9.640E−03 | 4.57E+06 |
| DOM7h-14-18 | 410 | 2.275E−01 | 5.60E+05 |
| DOM 7h-14-19 | 890 | 2.870E−01 | 3.20E+05 |
| DOM 7h-14-28 | 45 (140) | 7.0E−02 | 2.10E+06 |
| | | (1.141e−1) | (8.3e5) |
| DOM 7h-14-36 | 30 (6120) | 2.9E−02 | 1.55E+06 |
| | | (5.54e−2) | (9e3) |
| | Cyno | | |
| DOM 7h-14 | 66 | 9.65E−02 | 1.50E+06 |
| DOM 7h-14-10 | 9 | 1.15E−02 | 1.60E+06 |
| DOM 7h-14-18 | 180 | 1.05E−01 | 6.30E+5 |
| DOM 7h-14-19 | 225 | 1.56E−01 | 7.00E+05 |
| DOM 7h-14-28 | 66 (136) | 1.3E−01 | 2.50E+06 |
| | | (1.34e−1) | (9.8e5) |
| DOM 7h-14-36 | 35 (7830) | 1.9E−02 | 9.80E+06 |
| | | (1.1e−1) | (1.43e4) |
| | Mouse | | |
| DOM 7h-14 | 12 | 4.82E−02 | 4.10E+06 |
| DOM 7h-14-10 | 30 | 3.41E−02 | 1.29E+06 |
| DOM 7h-14-18 | 65 | 9.24E−02 | 2.28E+06 |
| DOM 7h-14-19 | 60 | 5.76E−02 | 1.16E+06 |
| DOM 7h-14-28 | 26 (31) | 3.4E−02 | 1.60E+06 |
| | | (7.15e−2) | (2.28e6) |
| DOM 7h-14-36 | 35 (33) | 2.3E−02 | 8.70E+05 |
| | | (7.06e−2) | (2.11e6) |
| | Human | | |
| DOM 7h-14 | 33 | 4.17E−02 | 1.43E+06 |
| DOM 7h-14-10 | 12 | 1.39E−02 | 1.50E+06 |
| DOM 7h-14-18 | 280 | 3.39E−02 | 1.89E+05 |
| DOM 7h-14-19 | 70 | 5.25E−02 | 8.26E+05 |
| DOM 7h-14-28 | 30 (8260) | 3.3E−02 | 1.24E+06 |
| | | (5.6e−2) | (6.78e3) |
| DOM 7h-14-36 | 28 (1260) | 2.4E−02 | 1.23E+06 |
| | | (6.7e−2) | (5.4e4) |

* values in brackets were derived from a second, independent SPR experiment.
All DOM7h-14 derived variants are cross-reactive to mouse, rat, human and cyno serum albumin. DOM7h-14-10 has improved affinity to rat, cyno and human serum albumin compared to parent. DOM7h-14-28 has an improved affinity to RSA. DOM7h-14-36 has an improved affinity to RSA, CSA and MSA.

### Example 3

### Origins of Key DOM7h-14 Lineage Clones

DOM7h-14-19: From affinity maturation performed against HSA using the error prone library, round 3 outputs (100 nM, HSA) with 100 ug/ml trypsin.

DOM7h-14-10, DOM7h-14-18, DOM7h-14-28, DOM7h-14-36: From affinity maturation performed against HSA using CDR3 library (Y92, Y93, T94, N96), round 3 output.

TABLE 5
CDR sequences(according to Kabat; ref. as above)

| | CDR | | |
|---|---|---|---|
| AlbudAb | CDR1 | CDR2 | CDR3 |
| DPK9 Vk dummy | SQSISSYLN | YAASSLQS | QQSYSTPNT |
| | (SEQ ID | (SEQ ID | (SEQ ID NO: 25) |
| | NO: 23) | NO: 24) | |
| DOM 7h-14 | SQWIGSQLS | MWRSSLQS | AQGAALPRT |
| | (SEQ ID | (SEQ ID | (SEQ ID NO: 28) |
| | NO: 26) | NO: 27) | |
| DOM 7h-14-10 | SQWIGSQLS | MWRSSLQS | AQGLRHPKT |
| | (SEQ ID | (SEQ ID | (SEQ ID NO: 31) |
| | NO: 29) | NO: 30) | |
| DOM 7h-14-18 | SQWIGSQLS | MWRSSLQS | AQGLMKPMT |
| | (SEQ ID | (SEQ ID | (SEQ ID NO: 34) |
| | NO: 32) | NO: 33) | |
| DOM 7h-14-19 | SQWIGSQLS | MWRSSLQS | AQGAALPRT |
| | (SEQ ID | (SEQ ID | (SEQ ID NO: 37) |
| | NO: 35) | NO: 36) | |
| DOM 7h-14-28 | SQWIGSQLS | MWRSSLQS | AQGAALPKT |
| | (SEQ ID | (SEQ ID | (SEQ ID NO: 40) |
| | NO: 38) | NO: 39) | |
| DOM 7h-14-36 | SQWIGSQLS | MWRSSLQS | AQGFKKPRT |
| | (SEQ ID | (SEQ ID | (SEQ ID NO: 43) |
| | NO: 41) | NO: 42) | |

### Example 4

### Expression and Biophysical Characterisation

The routine bacterial expression level in 2.5 L shake flasks was determined following culture in Onex media at 30° C. for 48 hrs at 250 rpm. The biophysical characteristics were determined by SEC MALLS and DSC.

SEC MALLS (size exclusion chromatography with multi-angle-LASER-light-scattering) is a non-invasive technique for the characterizing of macromolecules in solution. Briefly, proteins (at concentration of 1 mg/mL in buffer Dulbecco's PBS at 0.5 ml/min are separated according to their hydrodynamic properties by size exclusion chromatography (column: TSK3000 from TOSOH Biosciences; S200 from Pharmacia). Following separation, the propensity of the protein to scatter light is measured using a multi-angle-LASER-light-scattering (MALLS) detector. The intensity of the scattered light while protein passes through the detector is measured as a function of angle. This measurement taken together with the protein concentration determined using the refractive index (RI) detector allows calculation of the molar mass using appropriate equations (integral part of the analysis software Astra v.5.3.4.12).

DSC (Differential Scanning calorimetry): briefly, the protein is heated at a constant rate of 180° C./hrs (at 1 mg/mL in PBS) and a detectable heat change associated with thermal denaturation measured. The transition midpoint (ₐₚₚTₘ) is determined, which is described as the temperature where 50% of the protein is in its native conformation and the other 50% is denatured. Here, DSC determined the apparent transition midpoint (App™) as most of the proteins examined do not fully refold. The higher the Tm, the more stable the molecule. Unfolding curves were analysed by non-2-state equations. The software package used was Origin^{R} v7.0383.

TABLE 6

Biophysical parameters

| AlbudAb | SEC MALLS | DSC Tm(° C.) |
|---|---|---|
| DOM7h-14 | M | 60 |
| DOM 7h-14-10 | M | 59 |
| DOM 7h-14-18 | M | 58 |
| DOM 7h-14-19 | M | 59 |
| DOM 7h-14-28 | M | 58.3/60.2 |
| DOM 7h-14-36 | M | 59.2 |

* in one other trial, monomer was primarily seen by SEC MALLS, although lower than 95%

We observed expression levels for all clones in Table 6 in the range from 15 to 119 mg/L in E. coli.

For DOM7h-14 variants, favorable biophysical parameters (monomeric in solution as determined by SEC MALLs and App™ of >55° C. as determined by DSC) and expression levels were maintained during affinity maturation. Monomeric state is advantageous because it avoids dimerisation and the risk of products that may cross-link targets such as cell-surface receptors.

### Example 5

### Determination of Serum Half Life in Rat, Mouse and Cynomolgus Monkey

AlbudAbs DOM7h-14-10, DOM7h-14-18 and DOM7h-14-19, were cloned into the pDOM5 vector. For each AlbudAb™, 20-50 mg quantities were expressed in E. coli and purified from bacterial culture supernatant using protein L affinity resin and eluted with 100 mM glycine pH2. The proteins were concentrated to greater than 1 mg/ml, buffer exchanged into PBS and endotoxin depleted using Q spin columns (Vivascience). For Rat pharmacokinetic (PK) analysis, AlbudAbs were dosed as single i.v. injections at 2.5 mg/kg using 3 rats per compound. Serum samples were taken at 0.16, 1, 4, 12, 24, 48, 72, 120, 168 hrs. Analysis of serum levels was by anti-myc ELISA as per the method described below.

For Mouse PK dAbs were dosed as single i.v. injections at 2.5 mg/kg per dose group of 3 subjects and serum samples taken at 10 mins; 1 h; 8 h; 24 h; 48 h; 72 h; 96 h. Analysis of serum levels was by anti-myc ELISA as per the method described below.

For Cynomolgus monkey PK DOM7h-14-10 was dosed as single i.v. injections at 2.5 mg/kg into 3 female Cynomolgus monkeys per dose group and serum samples taken at 0.083, 0.25, 0.5, 1, 2, 4, 8, 24, 48, 96, 144, 192, 288, 336, 504 hrs. Analysis of serum levels was by anti-myc ELISA as per the method described below.

Anti-myc ELISA method

The AlbudAb concentration in serum was measured by anti-myc ELISA. Briefly, goat anti-myc polyclonal antibody (1:500; Abcam, catalogue number ab9132) was coated overnight onto Nunc 96-well Maxisorp plates and blocked with 5% BSA/PBS+1% Tween. Serum samples were added at a range of dilutions alongside a standard at known concentrations. Bound myc-tagged AlbudAb was then detected using a rabbit polyclonal anti-Vk (1:1000; in-house reagent, bleeds were pooled and protein A purified before use) followed by an anti-rabbit IgG HRP antibody (1:10,000; Sigma, catalogue number A2074). Plates were washed between each stage of the assay with 3×PBS+0.1% Tween20 followed by 3×PBS. TMB (SureBlue TMB 1-Component Microwell Peroxidase Substrate, KPL, catalogue number 52-00-00) was added after the last wash and was allowed to develop. This was stopped with 1M HCl and the signal was then measured using absorbance at 450 nm.

From the raw ELISA data, the concentration of unknown samples was established by interpolation against the standard curve taking into account dilution factors. The mean concentration result from each time point was determined from replicate values and entered into WinNonLin analysis package (e.g. version 5.1 (available from Pharsight Corp., Mountain View, Calif. 94040, USA). The data was fitted using a non-compartmental model, where PK parameters were estimated by the software to give terminal half-lives. Dosing information and time points were selected to reflect the terminal phase of each PK profile.

TABLE 7
Single AlbudAb ™ PK

| | | | PK parameters | | | |
|---|---|---|---|---|---|---|
| | | Albumin | AUC | CL | t½ | Vz |
| Species | AlbudAb | K_{D} (nM) | h × μg/ml | ml/h/kg | h | ml/kg |
| Rat | DOM7h-14* | 60 | | | | |
| | DOM7h-14-10 | 4 | 2134.6 | 1.2 | 42.1 | 71.2 |
| | DOM7h-14-18 | 410 | 617.3 | 4.1 | 38.4 | 228.1 |
| | DOM 7h-14-19 | 890 | 632.6 | 4.1 | 36.3 | 213.3 |
| Cyno | DOM 7h-14* | 66 | | | 217.5 | |
| | DOM 7h-14-10 | 9 | 6174.6 | 0.4 | 200.8 | 117.8 |

*Historical data

Pharmacokinetic parameters derived from rat, mouse and cynomolgus monkey studies were fitted using a non-compartmental model. Key: AUC: Area under the curve from dosing time extrapolated to infinity; CL: clearance; t½: is the time during which the blood concentration is halved; Vz: volume of distribution based on the terminal phase.

### Example 6

### AlbudAb™ IFN Fusions

### Cloning and Expression

As well as single AlbudAbs, the affinity matured Vk Albudabs were linked to Interferon alpha 2b (IFNα2b) to determine whether a useful PK of the AlbudAb was maintained as a fusion protein.

Interferon alpha 2b amino acid sequence:
(SEQ ID NO: 44)
CDLPQTHSLGSRRTLMLLAQMRRISLFSCLKDRHDFGFPQEEFGNQF
QKAETIPVLHEMIQQIFNLFSTKDSSAAWDETLLDKFYTELYQQLND
LEACVIQGVGVTETPLMKEDSILAVRKYFQRITLYLKEKKYSPCAWE
VVRAEIMRSFSLSTNLQESLRSKE
Interferon alpha 2b nucleotide sequence:
(SEQ ID NO: 45)
TGTGATCTGCCTCAAACCCACAGCCTGGGTAGCAGGAGGACCTTGAT
GCTCCTGGCACAGATGAGGAGAATCTCTCTTTTCTCCTGCTTGAAGG
ACAGACATGACTTTGGATTTCCCCAGGAGGAGTTTGGCAACCAGTTC
CAAAAGGCTGAAACCATCCCTGTCCTCCATGAGATGATCCAGCAGAT
CTTCAATCTCTTCAGCACAAAGGACTCATCTGCTGCTTGGGATGAGA
CCCTCCTAGACAAATTCTACACTGAACTCTACCAGCAGCTGAATGAC
CTGGAAGCCTGTGTGATACAGGGGGTGGGGGTGACAGAGACTCCCCT
GATGAAGGAGGACTCCATTCTGGCTGTGAGGAAATACTTCCAAAGAA
TCACTCTCTATCTGAAAGAGAAGAAATACAGCCCTTGTGCCTGGGAG
GTTGTCAGAGCAGAAATCATGAGATCTTTTTCTTTGTCAACAAACTT
GCAAGAAAGTTTAAGAAGTAAGGAA

IFNa2b was linked to the AlbudAb via a TVAAPS linker region (see WO2007085814). The constructs were cloned by SOE-PCR (single overlap extension according to the method of Horton et al. Gene, 77, p 61 (1989)). PCR amplification of the AlbudAb and IFN sequences were carried out separately using primers with a ˜15 base pair overlap at the TVAAPS linker region. The primers used are as follows:—

IFNα2b SOE fragment 5′
(SEQ ID NO: 46)
GCCCGGATCCACCGGCTGTGATCTG
IFNα2b SOE fragment 3′
(SEQ ID NO: 47)
GGAGGATGGAGACTGGGTCATCTGGATGTC
Vk SOE fragment 5′
(SEQ ID NO: 48)
GACATCCAGATGACCCAGTCTCCATCCTCCGCGCAAGCTTTTAT
TAATTCAGATCCTCTTC
Vk SOE fragment 3′
(SEQ ID NO: 49)
TGAGATGAGTTTTTGTTCTGCGGCCGCCCGTTTGATTTCCACCT
TGGTCCC
also introduce a myc tag

The fragments were purified separately and subsequently assembled in a SOE (single overlap extension PCR extension) reaction using only the flanking primers:

IFNα2b SOE fragment 5′
(SEQ ID NO: 50)
GCCCGGATCCACCGGCTGTGATCTGGCGCAAGCTTTTATTAAT
TCAGATCCTCTTC
Vk SOE fragment 3′ to
(SEQ ID NO: 51)
TGAGATGAGTTTTTGTTCTGCGGCCGCCCGTTTGATTTCCACC
TTGGTCCC
also introduce a myc tag

The assembled PCR product was digested using the restriction enzymes BamHI and HindIII and the gene ligated into the corresponding sites in the pDOM50, a mammalian expression vector which is a pTT5 derivative with an N-terminal V-J2-C mouse IgG secretory leader sequence to facilitate expression into the cell media.

Leader sequence (amino acid):
(SEQ ID NO: 52)
METDTLLLWVLLLWVPGSTG
Leader sequence (nucleotide):
(SEQ ID NO: 53)
ATGGAGACCGACACCCTGCTGCTGTGGGTGCTGCTGCTGT
GGGTGCCCGGATCCACCGGGC

Plasmid DNA was prepared using QIAfilter megaprep (Qiagen). 1 μg DNA/ml was transfected with 293-Fectin into HEK293E cells and grown in serum free media. The protein is expressed in culture for 5 days and purified from culture supernatant using protein L affinity resin and eluted with 100 mM glycine pH2. The proteins were concentrated to greater than 1 mg/ml, buffer exchanged into PBS and endotoxin depleted using Q spin columns (Vivascience).

TABLE 8
Interferon alpha 2b-AlbudAb sequences with and without myc-tag
(as amino acid-and nucleotide sequence)
The interferon alpha 2b is N-terminal to the AlbudAb in the following fusions

| | aa + myc | nt + myc | aa no tag | nt no tag |
|---|---|---|---|---|
| DMS7321 | CDLPQTHSLGSRRTLMLL | TGCGACTTGCCACAGACA | CDLPQTHSLGSRRTLMLL | TGCGACTTGCCACAGACA |
| (IFNα2b-DOM7h-14) | AQMRRISLFSCLKDRHDF | CATAGTTTGGGATCAAGA | AQMRRISLFSCLKDRHDF | CATAGTTTGGGATCAAGA |
| | GFPQEEFGNQFQKAETIP | AGAACATTGATGTTATTA | GFPQEEFGNQFQKAETIP | AGAACATTGATGTTATTA |
| | VLHEMIQQIFNLFSTKDS | GCACAAATGCGTAGAATT | VLHEMIQQIFNLFSTKDS | GCACAAATGCGTAGAATT |
| | SAAWDETLLDKFYTELQY | TCTTTGTTCTCTTGTCTA | SAAWDETLLDKFYTELYQ | TCTTTGTTCTCTTGTCTA |
| | QLNDLEACVIQGVGVTET | AAGGACCGTCACGACTTC | QLNDLEACVIQGVGVTET | AAGGACCGTCACGACTTC |
| | PLMKEDSILAVRKYFQRI | GGATTCCCTCAGGAAGAG | PLMKEDSILAVRKYFQRI | GGATTCCCTCAGGAAGAG |
| | TLYLKEKKYSPCAWEVVR | TTTGGAAACCAATTCCAA | TLYLKEKKYSPCAWEVVR | TTTGGAAACCAATTCCAA |
| | AEIMRSFSLSTNLQESLR | AAAGCAGAAACTATTCCT | AEIMRSFSLSTNLQESLR | AAAGCAGAAACTATTCCT |
| | SKETVAAPSDIQMTQSPS | GTCTTGCACGAAATGATC | SKETVAAPSDIQMTQSPS | GTCTTGCACGAAATGATC |
| | SLSASVGDRVTITCRASQ | CAGCAAATATTCAATTTG | SLSASVGDRVTITCRASQ | CAGCAAATATTCAATTTG |
| | WIGSQLSWYQQKPGKAPK | TTTTCTACAAAGGACTCA | WIGSQLSWYQQKPGKAPK | TTTTCTACAAAGGACTCA |
| | LLIMWRSSLQSGVPSRFS | TCAGCCGCTTGGGATGAA | LLIMWRSSLQSGVPSRFS | TCAGCCGCTTGGGATGAA |
| | GSGSGTDFTLTISSLQPE | ACTCTGTTAGATAAATTC | GSGSGTDFTLTISSLQPE | ACTCTGTTAGATAAATTC |
| | DFATYYCAQGAALPRTFG | TACACTGAACTATATCAA | DFATYYCAQGAALPRTFG | TACACTGAACTATATCAA |
| | QGTKVEIKRAAAEQKLIS | CAACTGAACGATCTAGAG | QGTKVEIKR | CAACTGAACGATCTAGAG |
| | EEDLN* | GCTTGCGTTATTCAGGGT | (SEQ ID NO: 56) | GCTTGCGTTATTCAGGGT |
| | (SEQ ID NO: 54) | GTAGGAGTTACTGAAACT | | GTAGGAGTTACTGAAACT |
| | | CCCCTAATGAAAGAAGAT | | CCCCTAATGAAAGAAGAT |
| | | TCAATTCTAGCCGTTAGA | | TCAATTCTAGCCGTTAGA |
| | | AAATACTTTCAGCGTATC | | AAATACTTTCAGCGTATC |
| | | ACATTGTATTTAAAGGAA | | ACATTGTATTTAAAGGAA |
| | | AAGAAATACTCCCCATGT | | AAGAAATACTCCCCATGT |
| | | GCATGGGAGGTGGTTAGA | | GCATGGGAGGTGGTTAGA |
| | | GCAGAAATTATGAGGTCC | | GCAGAAATTATGAGGTCC |
| | | TTCTCTCTTTCTACGAAT | | TTCTCTCTTTCTACGAAT |
| | | TTGCAAGAATCTTTGAGA | | TTGCAAGAATCTTTGAGA |
| | | TCTAAGGAAACCGTCGCT | | TCTAAGGAAACCGTCGCT |
| | | GCTCCATCTGACATCCAG | | GCTCCATCTGACATCCAG |
| | | ATGACCCAGTCTCCATCC | | ATGACCCAGTCTCCATCC |
| | | TCCCTGTCTGCATCTGTA | | TCCCTGTCTGCATCTGTA |
| | | GGAGACCGTGTCACCATC | | GGAGACCGTGTCACCATC |
| | | ACTTGCCGGGCAAGTCAG | | ACTTGCCGGGCAAGTCAG |
| | | TGGATTGGGTCTCAGTTA | | TGGATTGGGTCTCAGTT |
| | | TCTTGGTACCAGCAGAAA | | ATCTTGGTACCAGCAGA |
| | | CCAGGGAAAGCCCCTAAG | | AACCAGGGAAAGCCCCT |
| | | CTCCTGATCATGTGGCGT | | AAGCTCCTGATCATGTG |
| | | TCCTCGTTGCAAAGTGGG | | GCGTTCCTCGTTGCAAA |
| | | GTCCCATCACGTTTCAGT | | GTGGGGTCCCATCACGT |
| | | GGCAGTGGATCTGGGACA | | TTCAGTGGCAGTGGATC |
| | | GATTTCACTCTCACCATC | | TGGGACAGATTTCACTC |
| | | AGCAGTCTGCAACCTGAA | | TCACCATCAGCAGTCTG |
| | | GATTTTGCTACGTACTAC | | CAACCTGAAGATTTTGC |
| | | TGTGCTCAGGGTGCGGCG | | TACGTACTACTGTGCTC |
| | | TTGCCTAGGACGTTCGGC | | AGGGTGCGGCGTTGCCT |
| | | CAAGGGACCAAGGTGGAA | | AGGACGTTCGGCCAAGG |
| | | ATCAAACGGGCGGCCGCA | | GACCAAGGTGGAAATCA |
| | | GAACAAAAACTCATCTCA | | AACGG |
| | | GAAGAGGATCTGAATTAA | | (SEQ ID NO: 57) |
| | | (SEQ ID NO: 55) | | |
| DMS732 | CDLPQTHSLGSRRTLMLL | TGCGACTTGCCACAGACA | CDLPQTHSLGSRRTLMLL | TGCGACTTGCCACAGACA |
| (IFNα2b-DOM7h-14-10) | AQMRRISLFSCLKDRHDF | CATAGTTTGGGATCAAGA | AQMRRISLFSCLKDRHDF | CATAGTTTGGGATCAAGA |
| | GFPQEEFGNQFQKAETIP | AGAACATTGATGTTATTA | GFPQEEFGNQFQKAETIP | AGAACATTGATGTTATTA |
| | VLHEMIQQIFNLFSTKDS | GCACAAATGCGTAGAATT | VLHEMIQQIFNLFSTKDS | GCACAAATGCGTAGAATT |
| | SAAWDETLLDKTYTELYQ | TCTTTGTTCTCTTGTCTA | SAAWDETLLDKFYTELYQ | TCTTTGTTCTCTTGTCTA |
| | QLNDLEACIVIQGVGVTE | AAGGACCGTCACGACTTC | QLNDLEACVIQGVGVTET | AAGGACCGTCACGACTTC |
| | TPLMKEDSILAVRKYFQR | GGATTCCCTCAGGAAGAG | PLMKEDSILAVRKYFQRI | GGATTCCCTCAGGAAGAG |
| | ITLYLKEKKYSPCAWEVV | TTTGGAAACCAATTCCAA | TLYLKEKKYSPCAWEVVR | TTTGGAAACCAATTCCAA |
| | RAEIMRSFSLSTNLQESL | AAAGCAGAAACTATTCCT | AEIMRSFSLSTNLQESLR | AAAGCAGAAACTATTCCT |
| | RSKETVAAPSDIQMTQSP | GTCTTGCACGAAATGATC | SKETVAAPSDIQMTQSPS | GTCTTGCACGAAATGATC |
| | SSLSASVGDRVTITCRAS | CAGCAAATATTCAATTTG | SLSASVGDRVTITCRASQ | CAGCAAATATTCAATTTG |
| | QWIGSQLSWYQQKPGKAP | TTTTCTACAAAGGACTCA | WIGSQLSWYQQKPGKAPK | TTTTCTACAAAGGACTCA |
| | KLLIMWRSSLQSGVPSRF | TCAGCCGCTTGGGATGAA | LLIMWRSSLQSGVPSRFS | TCAGCCGCTTGGGATGAA |
| | SGSGSGTDFTLTISSLQP | ACTCTGTTAGATAAATTC | GSGSGTDFTLTISSLQPE | ACTCTGTTAGATAAATTC |
| | EDFATYYCAQGLRHPKTF | TACACTGAACTATATCAA | DFATYYCAQGLRHPKTFG | TACACTGAACTATATCAA |
| | GQGTKVEIKRAAAEQKLI | CAACTGAACGATCTAGAG | QGTKVEIKR | CAACTGAACGATCTAGAG |
| | SEEDLN* | GCTTGCGTTATTCAGGGT | (SEQ ID NO: 60) | GCTTGCGTTATTCAGGGT |
| | (SEQ ID NO: 58) | GTAGGAGTTACTGAAACT | | GTAGGAGTTACTGAAACT |
| | | CCCCTAATGAAAGAAGAT | | CCCCTAATGAAAGAAGAT |
| | | TCAATTCTAGCCTCAATT | | TCAATTCTAGCCGTTAGA |
| | | CTAGCCGTTAGAAAATAC | | AAATACTTTCAGCGTATC |
| | | TTTCAGCGTATCACATTG | | ACATTGTATTTAAAGGAA |
| | | TATTTAAAGGAAAAGAAA | | AAGAAATACTCCCCATGT |
| | | TACTCCCCATGTGCATGG | | GCATGGGAGGTGGTTAGA |
| | | GAGGTGGTTAGAGCAGAA | | GCAGAAATTATGAGGTCC |
| | | TTATGAGGTCCTTCTCTC | | TTCTCTCTTTCTACGAAT |
| | | TTTCTACGAATTTGCAAG | | TTGCAAGAATCTTTGAGA |
| | | AATCTTTGAGATCTAAGG | | TCTAAGGAAACCGTCGCT |
| | | AAACCGTCGCTGCTCCAT | | GCTCCATCTGACATCCAG |
| | | CTGACATCCAGATGACCC | | ATGACCCAGTCTCCATCC |
| | | AGTCTCCATCCTCCCTGT | | TCCCTGTCTGCATCTGTA |
| | | CTGCATCTGTAGGAGACC | | GGAGACCGTGTCACCATC |
| | | GTGTCACCATCACTTGCC | | ACTTGCCGGGCAAGTCAG |
| | | GGGCAAGTCAGTGGATTG | | TGGATTGGGTCTCAGTTA |
| | | GGTCTCAGTTATCTTGGT | | TCTTGGTACCAGCAGAAA |
| | | ACCAGCAGAAACCAGGGA | | CCAGGGAAAGCCCCTAAG |
| | | AAGCCCCTAAGCTCCTGA | | CTCCTGATCATGTGGCGT |
| | | TCATGTGGCGTTCCTCGT | | TCCTCGTTGCAAAGTGGG |
| | | TGCAAAGTGGGGTCCCAT | | GTCCCATCACGTTTCAGT |
| | | CACGTTTCAGTGGCAGTG | | GGCAGTGGATCTGGGACA |
| | | GATCTGGGACAGATTTCA | | GATTTCACTCTCACCATC |
| | | CTCTCACCATCAGCAGTC | | AGCAGTCTGCAACCTGAA |
| | | TGCAACCTGAAGATTTTG | | GATTTTGCTACGTACTAC |
| | | CTACGTACTACTGTGCTC | | TGTGCTCAGGGTTTGAGG |
| | | AGGGTTTGAGGCATCCTA | | CATCCTAAGACGTTCGGC |
| | | AGACGTTCGGCCAAGGGA | | CAAGGGACCAAGGTGGAA |
| | | CCAAGGTGGAAATCAAAC | | ATCAAACGG |
| | | GGGCGGCCGCAGAACAAA | | (SEQ ID NO: 61) |
| | | AACTCATCTCAGAAGAGG | | |
| | | ATCTGAATTAA | | |
| | | (SEQ ID NO: 59) | | |
| DMS7323 | CDLPQTHSLGSRRTLMLL | TGCGACTTGCCACAGACA | CDLPQTHSLGSRRTLMLL | TGCGACTTGCCACAGACA |
| (IFNα2b-DOM7h-14-18) | AQMRRISLFSCLKDRHDF | CATAGTTTGGGATCAAGA | AQMRRISLFSCLKDRHDF | CATAGTTTGGGATCAAGA |
| | GFPQEEFGNQFQKAETIP | AGAACATTGATGTTATTA | GFPQEEFGNQFQKAETIP | AGAACATTGATGTTATTA |
| | VLHEMIQQIFNLFSTKDS | GCACAAATGCGTAGAATT | VLHEMIQQIFNLFSTKDS | GCACAAATGCGTAGAATT |
| | SAAWDETLLDKFYTELYQ | TCTTTGTTCTCTTGTCTA | SAAWDETLLDKFYTELYQ | TCTTTGTTCTCTTGTCTA |
| | QLNDLEACVIQGVGVTET | AAGGACCGTCACGACTTC | QLNDLEACVIQGVGVTET | AAGGACCGTCACGACTTC |
| | PLMKEDSILAVRKYFQRI | GGATTCCCTCAGGAAGAG | PLMKEDSILAVRKYFQRT | GGATTCCCTCAGGAAGAG |
| | TLYLKEKKYSPCAWEVVR | TTTGGAAACCAATTCCAA | LYLKEKKYSPCAWEVVRA | TTTGGAAACCAATTCCAA |
| | AEIMRSFSLSTNLQESLR | AAAGCAGAAACTATTCCT | EIMRSFSLSTNLQESLRS | AAAGCAGAAACTATTCCT |
| | SKETVAAPSDIQMTQSPS | GTCTTGCACGAAATGATC | KETVAAPSDIQMTQSPSS | GTCTTGCACGAAATGATC |
| | SLSASVGDRVTITCRASQ | CAGCAAATATTCAATTTG | LSASVGDRVTITCRASQW | CAGCAAATATTCAATTTG |
| | WIGSQLSWYQQKPGKAPK | TTTTCTACAAAGGACTCA | IGSQLSWYQQKPGKAPKL | TTTTCTACAAAGGACTCA |
| | LLIMWRSSLQSGVPSRFS | TCAGCCGCTTTGGGATGA | LIMWRSSLQSGVPSRFSG | TCAGCCGCTTGGGATGAA |
| | GSGSGTDFTLTISSLQPE | AACTCTGTTAGATAAATT | SGSGTDFTLTISSLQPED | ACTCTGTTAGATAAATTC |
| | DFATYYCAQGLMKPMTFG | CTACACTGAACTATATCA | FATYYCAQGLMKPMTFG | TACACTGAACTATATCAA |
| | QGTKVEIKRAAAEQKLIS | ACAACTGAACGATCTAGA | QGTKVEIKR | CAACTGAACGATCTAGAG |
| | EEDLN* | GGCTTGCGTTATTCAGGG | (SEQ ID NO: 64) | GCTTGCGTTATTCAGGGT |
| | (SEQ ID NO: 62) | TGTAGGAGTTACTGAAAC | | GTAGGAGTTACTGAAACT |
| | | TCCCCTAATGAAAGAAGA | | CCCCTAATGAAAGAAGAT |
| | | TTCAATTCTAGCCGTTAG | | TCAATTCTAGCCGTTAGA |
| | | AAAATACTTTCAGCGTAT | | AAATACTTTCAGCGTATC |
| | | CACATTGTATTTAAAGGA | | ACATTGTATTTAAAGGAA |
| | | AAAGAAATACTCCCCATG | | AAGAAATACTCCCCATGT |
| | | TGCATGGGAGGTGGTTAG | | GCATGGGAGGTGGTTAGA |
| | | AGCAGAAATTATGAGGTC | | GCAGAAATTATGAGGTCC |
| | | TTCTCTCTTTCTACGAAT | | TTCTCTCTTTCTACGAAT |
| | | TTGCAAGAATCTTTGAGA | | TTGCAAGAATCTTTGAGA |
| | | TCTAAGGAAACCGTCGCT | | TCTAAGGAAACCGTCGCT |
| | | GCTCCATCTGACATCCAG | | GCTCCATCTGACATCCAG |
| | | ATGACCCAGTCTCCATCC | | ATGACCCAGTCTCCATCC |
| | | TCCCTGTCTGCATCTGTA | | TCCCTGTCTGCATCTGTA |
| | | GGAGACCGTGTCACCATC | | GGAGACCGTGTCACCATC |
| | | ACTTGCCGGGCAAGTCAG | | ACTTGCCGGGCAAGTCAG |
| | | TGGATTGGGTCTCAGTTA | | GGATTGGGTCTCAGTTAT |
| | | TCTTGGTACCAGCAGAAA | | CTTGGTACCAGCAGAAAC |
| | | CCAGGGAAAGCCCCTAAG | | CAGGGAAAGCCCCTAAGC |
| | | CTCCTGATCATGTGGCGT | | TCCTGATCATGTGGCGTT |
| | | TCCTCGTTGCAAAGTGGG | | CCTCGTTGCAAAGTGGGG |
| | | GTCCCATCACGTTTCAGT | | TCCCATCACGTTTCAGTG |
| | | GGCAGTGGATCTGGGACA | | GCAGTGGATCTGGGACAG |
| | | GATTTCACTCTCACCATC | | ATTTCACTCTCACCATCA |
| | | AGCAGTCTGCAACCTGAA | | GCAGTCTGCAACCTGAAG |
| | | GATTTTGCTACGTACTAC | | ATTTTGCTACGTACTACT |
| | | TGTGCTCAGGGTCTTATG | | GTGCTCAGGGTCTTATGA |
| | | AAGCCTATGACGTTCGGC | | AGCCTATGACGTTCGGCC |
| | | CAAGGGACCAAGGTGGAA | | AAGGGACCAAGGTGGAAA |
| | | ATCAAACGGGCGGCCGCA | | TCAAACGG |
| | | GAACAAAAACTCATCTCA | | (SEQ ID NO: 65) |
| | | GAAGAGGATCTGAATTAA | | |
| | | (SEQ ID NO: 63) | | |
| DMS7324 | CDLPQTHSLGSRRTLMLL | TGCGACTTGCCACAGACA | CDLPQTHSLGSRRTLMLL | TGCGACTTGCCACAGACA |
| (IFNα2b-DOM7h-14-19) | AQMRRISLFSCLKDRHDF | CATAGTTTGGGATCAAGA | AQMRRISLFSCLKDRHDF | CATAGTTTGGGATCAAGA |
| | GFPQEEFGMQFQKAETIP | AGAACATTGATGTTATTA | GFPQEEFGNQFQKAETIP | AGAACATTGATGTTATTA |
| | VLHEMIQQIFNLFSTKDS | GCACAAATGCGTAGAATT | VLHEMIQQIFNLFSTKDS | GCACAAATGCGTAGAATT |
| | SAAWDETLLDKFYTELYQ | TCTTTGTTCTCTTGTCTA | SAAWDETLLDKFYTELYQ | TCTTTGTTCTCTTGTCTA |
| | QLNDLEACVIQGVGVTET | AAGGACCGTCACGACTTC | QLNDLEACVIQGVGVTET | AAGGACCGTCACGACTTC |
| | PLMKEDSILAVRKYFQRI | GGATTCCCTCAGGAAGAG | PLMKEDSILAVRKYFQRI | GGATTCCCTCAGGAAGAG |
| | TLYLEKKYSPCAWEVVRA | TTTGGAAACCAATTCCAA | TLYLKEKKYSPCAWEVVR | TTTGGAAACCAATTCCAA |
| | EIMRSFSLSTNLQESLRS | AAAGCAGAAACTATTCCT | AEIMRSFSLSTNLQESLR | AAAGCAGAAACTATTCCT |
| | KETVAAPSDIQMTQSPSS | GTCTTGCACGAAATGATC | SKETVAAPSDIQMTQSPS | GTCTTGCACGAAATGATC |
| | LSASVGDRVTISCRASQW | CAGCAAATATTCAATTTG | SLSASVGDRVTISCRASQ | CAGCAAATATTCAATTTG |
| | IGSQLSWYQQKPGEAPKL | TTTTCTACAAAGGACTCA | WIGSQLSWYQQKPGEAPK | TTTTCTACAAAGGACTCA |
| | LIMWRSSLQSGVPSRFSG | TCAGCCGCTTGGGATGAA | LLIMWRSSLQSGVPSRFS | TCAGCCGCTTGGGATGAA |
| | SGSGTDFTLTISSLQPED | ACTCTGTTAGATAAATTC | GSGSGTDFTLTISSLQPE | ACTCTGTTAGATAAATTC |
| | FATYYCAQGAALPRTFGQ | TACACTGAACTATATCAA | DFATYYCAQGAALPRTFG | TACACTGAACTATATCAA |
| | GTKVEIKRAAAEQKLISE | CAACTGAACGATCTAGAG | QGTKVEIKR | CAACTGAACGATCTAGAG |
| | EDLN* | GCTTGCGTTATTCAGGGT | (SEQ ID NO: 68) | GCTTGCGTTATTCAGGGT |
| | (SEQ ID NO: 66) | GTAGGAGTTACTGAAACT | | GTAGGAGTTACTGAAACT |
| | | CCCCTAATGAAAGAAGAT | | CCCCTAATGAAAGAAGAT |
| | | TCAATTCTAGCCGTTAGA | | TCAATTCTAGCCGTTAGA |
| | | AAATACTTTCAGCGTATC | | AAATACTTTCAGCGTATC |
| | | ACATTGTATTTAAAGGAA | | ACATTGTATTTAAAGGAA |
| | | AAGAAATACTCCCCATGT | | AAGAAATACTCCCCATGT |
| | | GCATGGGAGGTGGTTAGA | | GCATGGGAGGTGGTTAGA |
| | | GCAGAAATTATGAGGTCC | | GCAGAAATTATGAGGTCC |
| | | TTCTCTCTTTCTACGAAT | | TTCTCTCTTTCTACGAAT |
| | | TTGCAAGAATCTTTGAGA | | TTGCAAGAATCTTTGAGA |
| | | TCTAAGGAAACCGTCGCT | | TCTAAGGAAACCGTCGCT |
| | | GCTCCATCTGACATCCAG | | GCTCCATCTGACATCCAG |
| | | ATGACCCAGTcTCCATCC | | ATGACCCAGTcTCCATCC |
| | | TCCCTGTCTGCATCTGTA | | TCCCTGTCTGCATCTGTA |
| | | GGAGACCGTGTCACCATC | | GGAGACCGTGTCACCATC |
| | | TCTTGCCGGGCAAGTCAG | | TCTTGCCGGGCAAGTCAG |
| | | TGGATTGGGTCTCAGTTA | | TGGATTGGGTCTCAGTTA |
| | | TCTTGGTACCAGCAGAAA | | TCTTGGTACCAGCAGAAA |
| | | CCAGGGGAAGCCCCTAAG | | CCAGGGGAAGCCCCTAAG |
| | | CTCCTGATCATGTGGCGT | | CTCCTGATCATGTGGCGT |
| | | TCCTCGTTGCAAAGTGGG | | TCCTCGTTGCAAAGTGGG |
| | | GTCCCATCACGTTTCAGT | | GTCCCATCACGTTTCAGT |
| | | GGCAGTGGATCTGGGACA | | GGCAGTGGATCTGGGACA |
| | | GATTTCACTCTCACCATC | | GATTTCACTCTCACCATC |
| | | AGCAGTCTGCAACCTGAA | | AGCAGTCTGCAACCTGAA |
| | | GATTTTGCTACGTACTAC | | GATTTTGCTACGTACTAC |
| | | TGTGCTCAGGGTGCGGCG | | TGTGCTCAGGGTGCGGCG |
| | | TTGCCTAGGACGTTCGGC | | TTGCCTAGGACGTTCGGC |
| | | CAAGGGACCAAGGTGGAA | | CAAGGGACCAAGGTGGAA |
| | | ATCAAACGGGCGGCCGCA | | ATCAAACCG |
| | | GAACAAAAACTCATCTCA | | (SEQ ID NO: 69) |
| | | GAAGAGGATCTGAATTA | | |
| | | (SEQ ID NO: 67) | | |

The amino acid and nucleotide sequences highlighted in bold represents the cloning
site and MYC tag.
*represents the stop codon at the end of the gene.

### Affinity Determination and Biophysical Characterisation:

To determine the binding affinity (K_{D}) of the AlbudAb-IFNα2b fusion proteins to each serum albumin; purified fusion proteins were analysed by BIAcore over albumin (immobilised by primary-amine coupling onto CM5 chips; BIAcore) using fusion protein concentrations from 5000 nM to 39 nM (5000 nM, 2500 nM, 1250 nM, 625 nM, 312 nM, 156 nM, 78 nM, 39 nM) in HBS-EP BIAcore buffer.

TABLE 9
Affinity to SA

| | | Affinity to | | |
|---|---|---|---|---|
| AlbudAb | Fusion | SA (nM) | Kd | Ka |
| | | Rat | | |
| DOM7h-14 | IFNα2b | 350 | 4.500E−02 | 1.28E+05 |
| DOM7h-14-10 | IFNα2b | 16 | 4.970E−03 | 5.90E+05 |
| DOM 7h-14-18 | IFNα2b | 780 | 2.127E−01 | 5.80E+05 |
| DOM 7h-14-19 | IFNα2b | 1900 | 1.206E−01 | 7.96E+04 |
| | | Cyno | | |
| DOM 7h-14 | IFNα2b | 60 | 1.32E−02 | 5.0E+05 |
| DOM 7h-14-10 | IFNα2b | 19 | 7.05E−03 | 4.50E+05 |
| DOM 7h-14-18 | IFNα2b | no binding | no binding | no binding |
| DOM 7h-14-19 | IFNα2b | 520 | 8.47E−02 | 2.73E+05 |
| | | Mouse | | |
| DOM 7h-14 | IFNα2b | 240 | 3.21E−02 | 1.50E+06 |
| DOM 7h-14-10 | IFNα2b | 60 | 3.45E−02 | 6.86E+05 |
| DOM 7h-14-18 | IFNα2b | 180 | 1.50E−01 | 9.84E+05 |
| DOM 7h-14-19 | IFNα2b | 490 | 4.03E−02 | 1.19E+05 |
| | | Human | | |
| DOM 7h-14 | IFNα2b | 244 | 2.21E−02 | 9.89E+04 |
| DOM 7h-14-10 | IFNα2b | 32 | 6.58E−03 | 3.48E+05 |
| DOM 7h-14-18 | IFNα2b | 470 | 2.75E−01 | 6.15E+05 |
| DOM 7h-14-19 | IFNα2b | 350 | 4.19E−02 | 1.55E+05 |

When IFNα2b is linked to the AlbudAb variants, in all cases the affinity of AlbudAb binding to serum albumin is reduced. DOM7h-14-10 retains improved binding affinity to serum albumin across species compared to parent.

TABLE 10
Biophysical Characterisation
Biophysical Characterisation was carried out by SEC MALLS
and DSC as described above for the single AlbudAbs.

| | | DMS | Biophysical parameters | |
|---|---|---|---|---|
| AlbudAb | Fusion | number | SEC MALLS | DSC Tm(° C.) |
| DOM 7h-14 | IFNα2b | DMS7321 | M/D | 58-65 |
| DOM 7h-14-10 | IFNα2b | DMS7322 | M/D | 55-65 |
| DOM 7h-14-18 | IFNα2b | DMS7323 | M/D | 55-65 |
| DOM 7h-14-19 | IFNα2b | DMS7324 | M/D | 59-66 |

M/D indicates a monomer/dimer equilibrium as detected by SEC MALLS

We observed expression for all clones in Table 10 in the range of 17.5 to 54 mg/L in HEK293.

For IFNα2b-DOM7h-14 variants, favorable biophysical parameters and expression levels were maintained during affinity maturation.

PK Determination for AlbudAb-IFNα2bfusions

AlbudAbs IFNα2b fusions DMS7321 (IFNα2b-DOM7h-14) DMS7322 (IFNα2b-DOM7h-14-10) DMS7323 (IFNα2b-DOM7h-14-18), DMS7324 (IFNα2b-DOM7h-14-19), were expressed with the myc tag at 20-50 mg quantities in HEK293 cells and purified from culture supernatant using protein L affinity resin and eluted with 100 mM glycine pH2. The proteins were concentrated to greater than 1 mg/ml, buffer exchanged into Dulbecco's PBS and endotoxin depleted using Q spin columns (Vivascience).

For Rat PK, IFN-AlbudAbs were dosed as single i.v. injections at 2.0 mg/kg using 3 rats per compound. Serum samples were taken at 0.16, 1, 4, 8, 24, 48, 72, 120, 168 hrs. Analysis of serum levels was by EASY ELISA according to manufacturer's instructions (GE Healthcare, catalogue number RPN5960).

For Mouse PK, DMS7322 (IFN2b-DOM7h-14-10) with myc tag was dosed as single i.v. injections at 2.0 mg/kg per dose group of 3 subjects and serum samples taken at 10 mins; 1 h; 8 h; 24 h; 48 h; 72 h; 96 h. Analysis of serum levels was by EASY ELISA according to manufacturer's instructions (GE Healthcare, catalogue number RPN5960).

TABLE 11

| | | | | PK parameters | | | |
|---|---|---|---|---|---|---|---|
| | | | | | AUC | CL | |
| | | | Albumin | | h × | ml/ | Vz |
| Species | AlbudAb | Fusion | K_{D} (nM) | ug/ml | h/kg | t½ h | ml/kg |
| Rat | 7h-14 | IFNα2b | 350 | 832.1 | 2.4 | 27 | 94.5 |
| | 7h-14-10 | IFNα2b | 16 | 1380.7 | 1.5 | 35.8 | 75.2 |
| | 7h-14-18 | IFNα2b | 780 | 691.2 | 2.9 | 22.4 | 93.7 |
| | 7h-14-19 | IFNα2b | 1900 | 969.4 | 2.2 | 25 | 78.7 |
| Mouse | 7h-14 | IFNα2b | 240 | 761.2 | 2.6 | 30.4 | 115.3 |
| | 7h-14-10 | IFNα2b | 60 | 750.5 | 2.7 | 30.9 | 118.6 |

Pharmacokinetic parameters derived from rat and mouse studies were fitted using a non-compartmental model. Key: AUC: Area under the curve from dosing time extrapolated to infinity; CL: clearance; t½: is the time during which the blood concentration is halved; Vz: volume of distribution based on the terminal phase.

IFNα2b—AlbudAbs were tested in rat and mouse. The improvement in t½ correlates with the improved in vitro K_{D} to serum albumin. For IFNα2b-DOM7h-14-10 variants, the improvement in in vitro K_{D} to serum albumin also correlated to an improvement in t½ in rat.

All IFNα2b—AlbudAb fusion proteins exhibit a 5 to 10-fold decrease in the binding to RSA compared to the single AlbudAb.

### Example 7

### Further AlbudAb Fusions with Proteins, Peptides and NCEs

Various AlbudAbs fused to other chemical entities namely domain antibodies (dAbs), peptides and NCEs were tested. The results are shown in Table 12.

TABLE 12

| | | | | PK parameters | | | |
|---|---|---|---|---|---|---|---|
| | | | Albumin | AUC | CL | | Vz |
| Species | AlbudAb | Fusion | K_{D} (nM) | h × ug/ml | ml/h/kg | t½ h | ml/kg |
| Rat | DOM7h-14 | Exendin-4 | 2400 | 18 | 57.1 | 11 | 901.9 |
| | DOM7h-14- | Exendin-4 | 19 | 43.6 | 23.1 | 22.1 | 740.3 |
| | 10 | | | | | | |
| | DOM7h-14- | Exendin-4 | 16000 | 16.9 | 75.7 | 9.4 | 1002.5 |
| | 18 | | | | | | |
| | DOM7h-14- | Exendin-4 | 17000 | 31.4 | 32.5 | 11.9 | 556.7 |
| | 19 | | | | | | |
| | DOM7h14- | NCE- | 62 | | | | |
| | 10 | GGGGSC | | | | | |
| | DOM7h14- | NCE- | 35 | | | | |
| | 10 | TVAAPSC | | | | | |
| Human | DOM7h-14 | NCE | 204 | | | | |

Key:
DOM1m-21-23 is an anti-TNFR1 dAb, Exendin-4 is a peptide (a GLP-1 agonist) of 39 amino acids length. NCE, NCE-GGGGSC and NCE-TVAAPSC are described below.

Previously we have described the use of genetic fusions with an albumin-binding dAb (AlbudAb) to extend the PK half-life of anti-TNFR1 dAbs in vivo (see, e.g., WO04003019, WO2006038027, WO2008149148). Reference is made to the protocols in these PCT applications. In the table above, DOM1m-21-23 is an anti-mouse TNFR1 dAb.

To produce genetic fusions of exendin-4 or with DOM7h-14 (or other AlbudAb) which binds serum albumin, the exendin-4-linker-AlbudAb sequence was cloned into the pTT-5 vector (obtainable from CNRC, Canada). In each case the exendin-4 was at the 5′ end of the construct and the dAb at the 3′ end. The linker was a (G₄S)₃ linker. Endotoxin-free DNA was prepared in E. coli using alkaline lysis (using the endotoxin-free plasmid Giga kit, obtainable from Qiagen CA) and used to transfect HEK293E cells (obtainable from CNRC, Canada). Transfection was into 250 ml/flask of HEK293E cells at 1.75×10⁶ cells/ml using 333 ul of 293fectin (Invitrogen) and 250 ug of DNA per flask and expression was at 30° C. for 5 days. The supernatant was harvested by centrifugation and purification was by affinity purification on protein L. Protein was batch bound to the resin, packed on a column and washed with 10 column volumes of PBS. Protein was eluted with 50 ml of 0.1M glycine pH2 and neutralized with Tris pH8. Protein of the expected size was identified on an SDS-PAGE gel.

### NCE Albudab Fusions:

A new chemical entity (NCE) AlbudAb fusion was tested. The NCE, a small molecule ADAMTS-4 inhibitor was synthesised with a PEG linker (PEG 4 linker (i.e. 4 PEG molecules before the maleimide) and a maleimide group for conjugation to the AlbudAb. Conjugation of the NCE to the AlbudAb is via an engineered cysteine residue at amino acid position R108c, or following a 5 amino acid (GGGGSC) or 6 amino acid (TVAAPSC) spacer engineered at the end of the AlbudAb. Briefly, the AlbudAb was reduced with TCEP (Pierce, Catalogue Number 77720), desalted using a PD10 column (GE healthcare) into 25 mM Bis-Tris, 5 mM EDTA, 10% (v/v) glycerol pH6.5. A 5 fold molar excess of maleimide activated NCE was added in DMSO not to exceed 10% (V/V) final concentration. The reaction was incubated over night at room temperature and dialysed extensively into 20 mM Tris pH7.4

### PEG Linker:

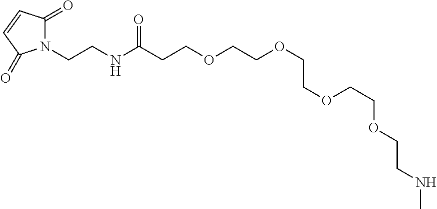 [SMILES: CNCCOCCOCCOCCOCCC(=O)NCCN1C(=O)C=CC1=O]

Sequences:
DOM7h-14 R108C:
(SEQ ID NO: 70)
DIQMTQSPSSLSASVGDRVTITCRASQWIGSQLSWYQQKPGKAPKLL
IMWRSSLQSGVPSRFSGSGSGTDFTLTISSLQPEDFATYYCAQGLRH
PKTFGQGTKVEIKC
Nucleotide:
(SEQ ID NO: 71)
GACATCCAGATGACCCAGTCTCCATCCTCCCTGTCTGCATCTGTAGG
AGACCGTGTCACCATCACTTGCCGGGCAAGTCAGTGGATTGGGTCTC
AGTTATCTTGGTACCAGCAGAAACCAGGGAAAGCCCCTAAGCTCCTG
ATCATGTGGCGTTCCTCGTTGCAAAGTGGGGTCCCATCACGTTTCAG
TGGCAGTGGATCTGGGACAGATTTCACTCTCACCATCAGCAGTCTGC
AACCTGAAGATTTTGCTACGTACTACTGTGCTCAGGGTTTGAGGCAT
CCTAAGACGTTCGGCCAAGGGACCAAGGTGGAAATCAAATGC

See Table 3 for the sequences of DOM7h-14-10/TVAAPSC and DOM7h-14-10/GGGGSC (ie, DOM7h-14-10/G4SC).

NCE-AlbudAbs DOM7h-14-10 GGGGSC and DOM7h14-10 TVAAPSC, exhibit a 5 to 10 fold decrease in in vitro affinity (K_{D}) to RSA as determined by BIAcore when fused to the chemical entity. PK data are not available for these molecules yet.

Exendin 4-AlbudAb fusion: the effect of fusing the AlbudAbs to a peptide on the binding ability to RSA is about 10-fold, apart from DOM7h-14-10, which only shows a 4-fold decrease in binding.

For all the above data, the T½ of the fusion increased with improved affinity to the species' SA.

We generally classify Albudab-therapeutics as being therapeutically amenable (for treatment and/or prophylaxis of diseases, conditions or indications) when the AlbudAb-drug fusions show an affinity range (K_{D}) of from 0.1 nM to 10 mM for serum albumin binding.

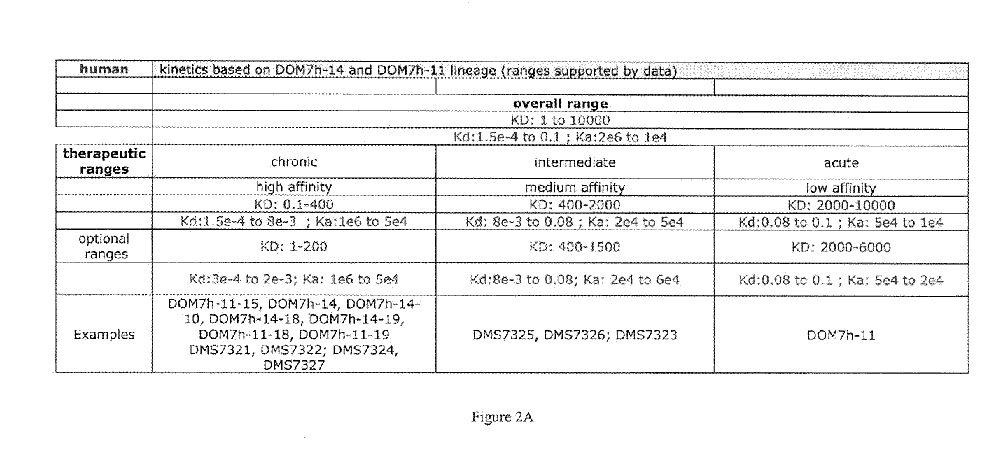

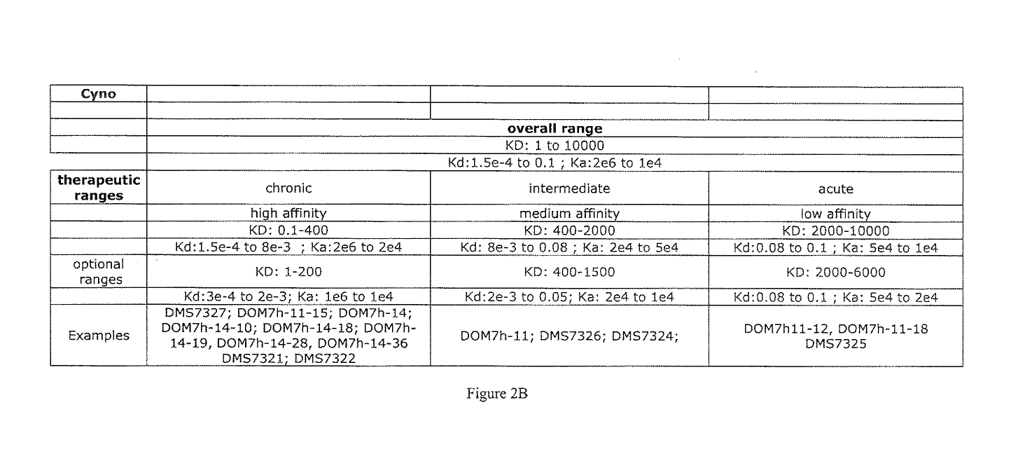

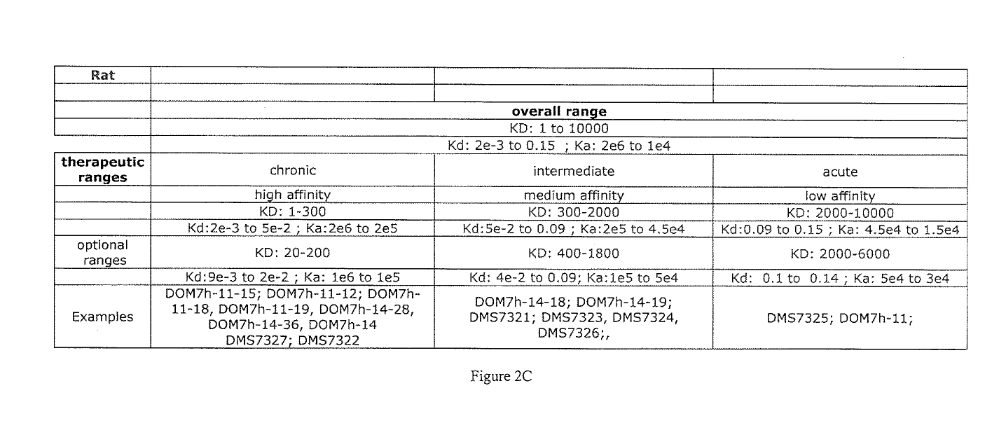

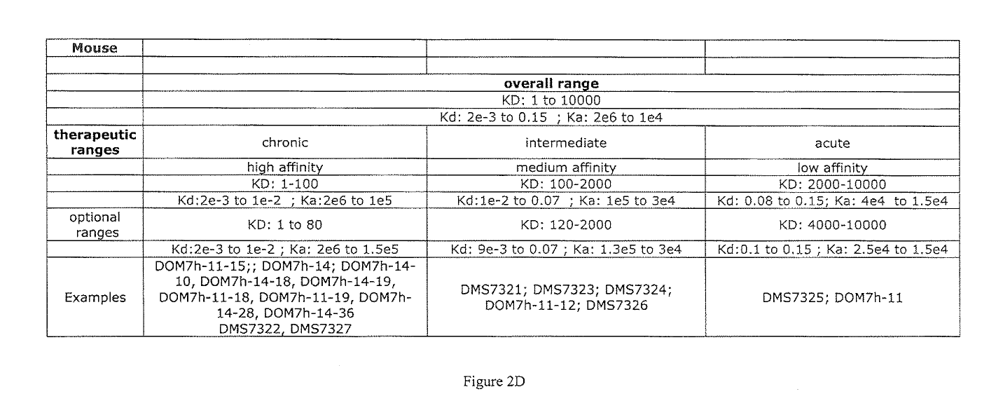

We define the therapeutic ranges of AlbudAbs and AlbudAb fusions (Protein-AlbudAbs for example IFNα2b-DOM7h-14-10; Peptide-AlbudAbs for example Exendin-4-DOM7h-14-10; dAb-AlbudAbs for example DOM1m21-23-DOM7h11-15; NCE-AlbudAb for example ADAMTS-4-DOM7h-14-10) as follows: Affinity (K_{D}) ranges that are useful for therapy of chronic or acute conditions, diseases or indications are shown. Also shown are affinity ranges marked as “intermediate”. AlbudAbs and fusions in this range have utility for chronic or acute diseases, conditions or indications. In this way, the affinity of the AlbudAb or fusion for serum albumin can be tailored or chosen according to the disease, condition or indication to be addressed. As described above, the invention provides AlbudAbs with affinities that allow for each AlbudAb to be categorised as “high affinity”, “medium affinity” or “low affinity”, thus enabling the skilled person to select the appropriate AlbudAb of the invention according to the therapy at hand. See FIG. 2.

### Example 8

### Improved Single Variable Domains

Affinity maturation of DOM7h-14-10 was performed and new variants were selected on the basis of specific binding to serum albumin from various species (human, Cynomolgous monkey, rat and mouse).

### Selections:

HSA (Human Serum Albumin) and RSA (Rat Serum Albumin) antigens and biotinylated products were obtained as described in Example 1.

### Affinity Maturation Libraries:

Both error prone and doped libraries were created using DOM7h-14-10 parental dAb (see SEQ ID NO: 2) as a template with arginine at position 108 mutated to tryptophan (DOM7h-14-10 R108W) allowing use of trypsin for phage selection. The libraries were generated in the pDOM33 vector.

For the doped CDR libraries, primary PCR reactions were performed using doped oligonucleotides containing biased degenerated codons to diversify the required positions in the dAb. Generation of doped libraries is described, for example, in Balint and Larrick, Gene, 137, 109-118 (1993). Primers were designed in order to change only the first two nucleotides from each degenerated codon so that the parental nucleotides were present in 85% of cases and in 5% of cases all other possible nucleotides were present. Six codons per CDR were targeted for being mutated simultaneously with 15% probability per nucleotide in the codon to be different than the parental nucleotide.

Assembly PCR was then used to generate a full length diversified insert. The inserts were digested with Sal I and Not I and used in a ligation reaction with pDOM33. The ligation of libraries were then used to transform E. coli strain TB1 by electroporation and the transformed cells plated on 2×TY agar containing 15 μg/ml tetracycline.

i) Selection Strategies: Selections Against HSA

Two rounds of selection against HSA were carried out. Each CDR library was selected as an individual pool in all rounds. Both rounds of selections were performed in solution against biotinylated HSA at 10 nM concentration. Libraries were eluted with 0.1M glycine pH 2.0 before neutralization with 1M Tris pH 8.0 and before infection into log phase TG1 cells. The second round of each selection was subcloned into pDOM5 for screening. Cross over selection Two rounds of selection against biotinylated SA in solution were carried out. Two rounds of selection performed with HSA (10 nM, 1 nM) and RSA (25 nM, 10 nM, 1 nM) in different orders, with or without trypsin treatment. Each CDR library was selected as an individual pool in all rounds. Libraries were eluted with 0.1M glycine pH 2.0 before neutralization with 1M Tris pH 8.0 and before infection into log phase TG1 cells. The second round of each selection was subcloned into pDOM5 for screening.

Ii) Screening Strategy and Affinity Determination

In each case after selection a pool of phage DNA from the appropriate round of selection was prepared using a QIAfilter midiprep kit (Qiagen), the DNA is digested using the restriction enzymes Sal1 and Not1 and the enriched V genes are ligated into the corresponding sites in pDOM5 the soluble expression vector which expresses the dAb with a myc tag (see PCT/EP2008/067789). The ligated DNA is used to transform chemically competent E. coli HB 2151 cells which are then grown overnight on agar plates containing the antibiotic carbenicillin. The resulting colonies are individually assessed for antigen binding. For each selection output, 93 clones were tested for binding to HSA, and RSA by BIAcore™ (surface plasmon resonance). Soluble dAb fragments were produced in bacterial culture in ONEX culture media (Novagen) overnight at 37° C. in 96 well plates. The culture supernatant containing soluble dAb was centrifuged and analysed by BIAcore for binding to high density HSA, and RSA CM5 chips. Clones which were found to bind equally or better than parental clone to both these species of serum albumin by off-rate screening were sequenced revealing unique dAb sequences.

Sequence homology to the parental sequences is shown below in Table 13.

TABLE 13

| | DOM7h- | DOM7h- | DOM7h- | DOM7h- | DOM7h- | DOM7h- | DOM7h- |
|---|---|---|---|---|---|---|---|
| | 14- | 14- | 14- | 14- | 14- | 14- | 14- |
| | 56 | 65 | 74 | 76 | 82 | 100 | 101 |
| DOM7h- | 0.972 | 0.981 | 0.962 | 0.972 | 0.981 | 0.972 | 0.972 |
| 14-10 | | | | | | | |

| | DOM7h- | DOM7h- | DOM7h- | DOM7h- | DOM7h- | DOM7h- | DOM7h- | DOM7h- |
|---|---|---|---|---|---|---|---|---|
| | 14- | 14- | 14- | 14- | 14- | 14- | 14- | 14- |
| | 109 | 115 | 116 | 119 | 120 | 121 | 122 | 123 |
| DOM7h- | 0.962 | 0.972 | 0.972 | 0.99 | 0.981 | 0.99 | 0.981 | 0.972 |
| 14-10 | | | | | | | | |

value * 100 = % sequence homology

Unique dAbs were expressed as bacterial supernatants in 0.5 L shake flasks in Onex media at 30° C. for 48 hrs at 250 rpm. dAbs were purified from the culture media by absorption to protein L streamline followed by elution with 100 mM glycine pH2.0. To determine the binding affinity (K_{D}) of the AlbudAbs to Human, Rat, Mouse and Cynomolgus serum albumin; purified dAbs were analysed by BIAcore over albumin concentration range from 500 nM to 3.9 nM (500 nM, 250 nM, 125 nM, 31.25 nM, 15.625 nM, 7.8125 nM, 3.90625 nM).

MSA antigen was obtained from Sigma (essentially fatty acid free, ˜99% (agarose gel electrophoresis), lyophilized powder Cat. No. A3559) and CSA was purified from Cynomolgus serum albumin using prometic blue resin (Amersham).

The affinities to all tested serum albumin species of key clones is presented in Table 14.

iii) Expression and Biophysical Characterisation:

Bacterial expression and characterisation by SECMALLS and DSC was carried out as described above in Example 4.

T/D and D/M indicates an equilibrium between trimer and dimer or dimer and monomer, respectively, as detected by SEC-MALLS.

TABLE 14
Characteristics of DOM7h-14-10 variants

| | | | | | Average | | |
|---|---|---|---|---|---|---|---|
| | RSA | HSA | CSA | MSA | expression | Thermal | |
| | KD | KD | KD | KD | level | stability | Solution |
| | (nM) | (nM) | (nM) | (nM) | (mg/ml) | Tm (° C.) | state |
| DOM7h-14-10 | 9.4 | 10.3 | 13.6 | 30.1 | 22 | 54.3 | T/D, |
| | | | | | | | Monomer |
| DOM7h-14-56 | 3.4 | 11.1 | 18.7 | 6.3 | 12 | 55.6 | T/D, |
| | | | | | | | Monomer |
| DOM7h-14-65 | 3.4/25* | 4.5 | 6.0/16* | 15.3 | 14 | 54.8 | D/M, |
| | | | | | | | Monomer |
| DOM7h-14-74 | 4.3/11.6* | 4.8 | 6.3/2.7* | 23.5 | 26 | 53.5 | T/D, D/M |
| DOM7h-14-76 | 4.7 | 6.3 | 19.9 | 11.5 | 6 | 52.7 | D/M, |
| | | | | | | | Monomer |
| DOM7h-14-82 | 11.8 | 15.4 | 170.5 | 32.3 | 13 | 54.1 | Dimer, |
| | | | | | | | Monomer |
| DOM7h-14-100 | 3.7/29* | 4.5 | 6.4/6.1* | 8.0 | 26 | 54.4 | Monomer |
| DOM7h-14-101 | 5.8 | 8.4 | 11.6 | 16.8 | 9 | 54.5 | Monomer |
| DOM7h-14-109 | 15.4 | 107.6 | 17.6 | 58.1 | 33 | 54.3 | D/M, |
| | | | | | | | Monomer |
| DOM7h-14-115 | 3.3 | 5.6 | 25.4 | 9.8 | 3.7 | 55.2 | Monomer |
| DOM74-14-116 | 8.5 | 7.0 | 22.8 | 19.1 | 9.6 | 54.7 | D/M, |
| | | | | | | | Monomer |

data for clones with W108R

| | | | | | | | |
|---|---|---|---|---|---|---|---|
| DOM7h-14-119 | 6.2 | 16.1 | 4.3 | ND | 10.7 | 56.1 | Monomer |
| DOM7h-14-120 | 2.7 | 14 | 3.7 | ND | 6.3 | 57.1 | Monomer |
| DOM7h-14-121 | 11.7 | 41.4 | 18.2 | ND | 8.7 | 51.7 | Monomer |
| DOM7h-14-122 | 7.8 | 43.4 | 9.8 | ND | 6.5 | 53 | Monomer |
| DOM7h-14-123 | 5.1 | 12 | 8.0 | ND | 8.3 | 56.6 | D/M, |
| | | | | | | | Monomer |

*second value originates from the analysis of a second protein batch by a second analyst.
M = monomer,
D = Dimer,
T = Trimer;
ND = not determined
DOM7h-14-100 has single-digit nM KD across the species tested. DOM7h-14-100 beneficially also is a monomer in solution.

Amino acid and nucleotide sequences are listed below. Most of the clones have arginine at position 108 mutated to tryptophan which was done to enable trypsin driven selection if necessary (knocking trypsin recognition site out)—this mutation was not crucial for AlbudAb binding to serum albumin.

Other clones (see DOM7h-14-119, DOM7h-14-120, DOM7h-14-121, DOM7h-14-122, DOM7h-14-122) were derived in which position 108 was back mutated to arginine (W108R) and, optionally, position 106 was back mutated to isoleucine. The sequences of these clones are listed below.

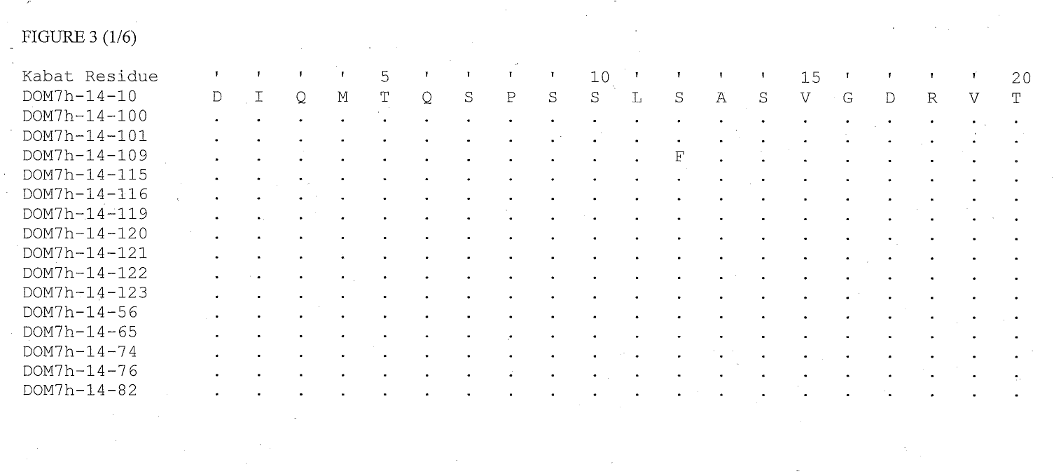

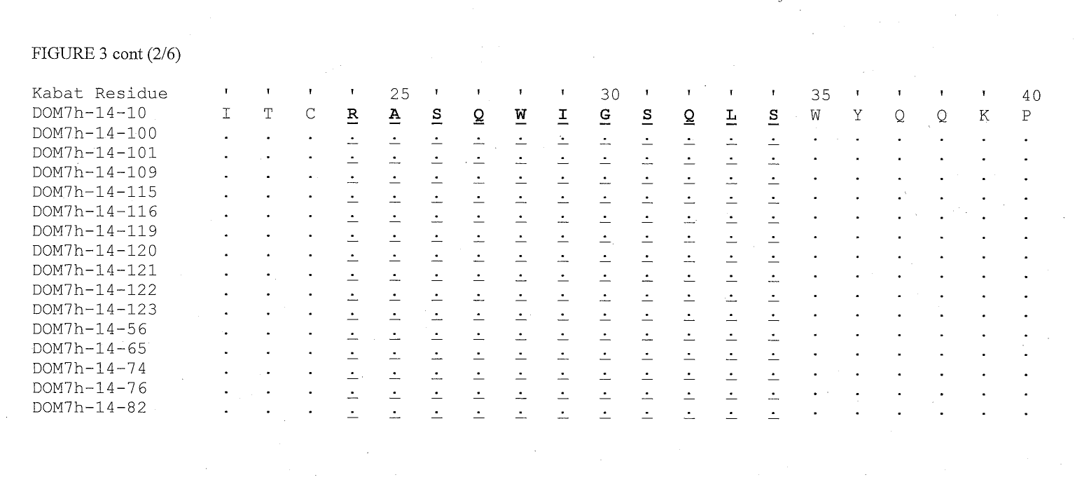

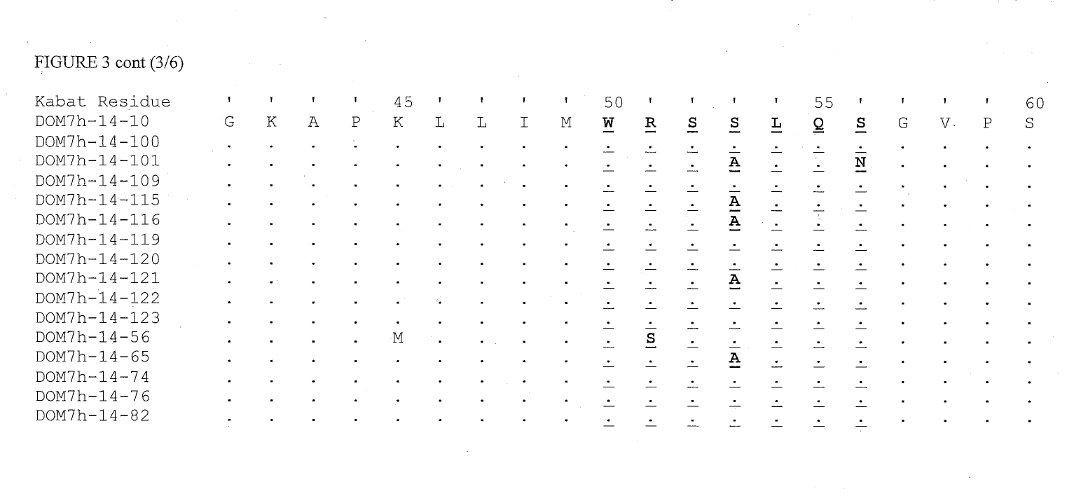

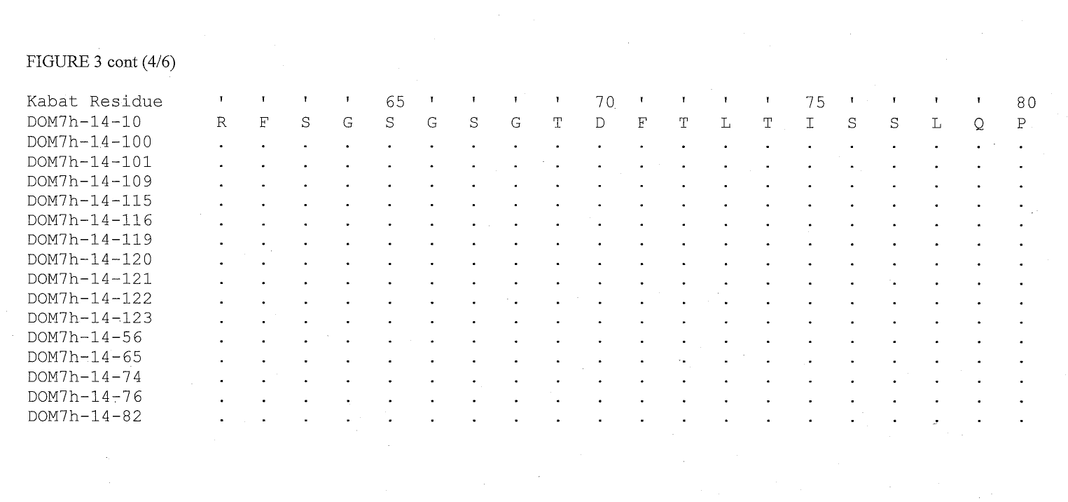

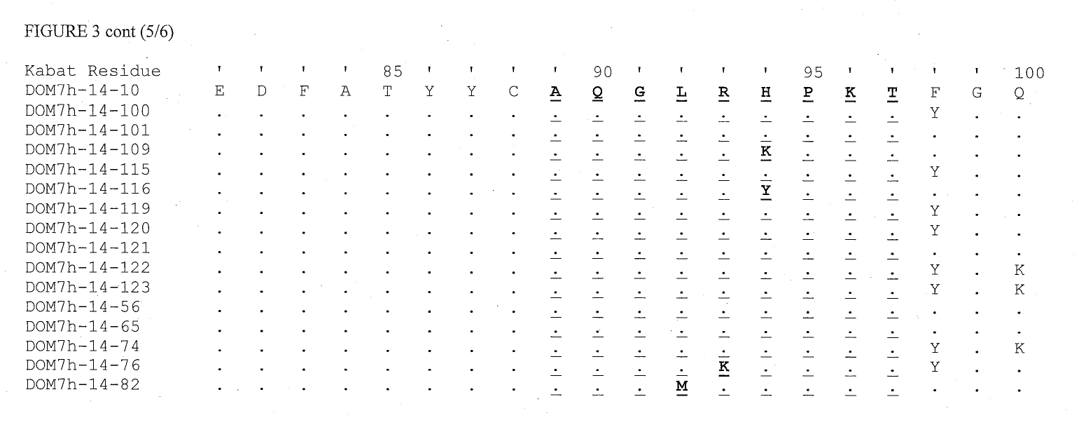

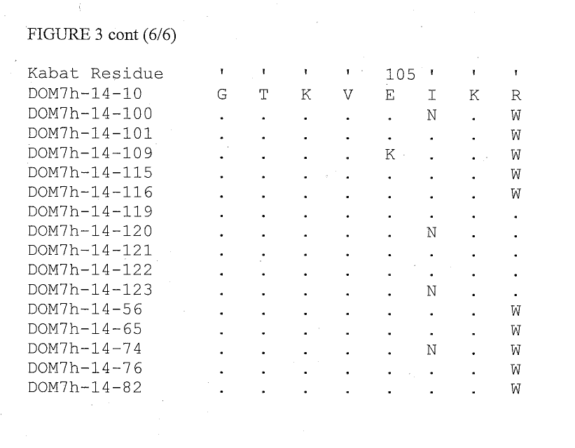

A sequence alignment is shown in FIG. 3.

TABLE 15
Amino Acid sequences of DOM7h-14-10 variants

DOM7h-14-56
(SEQ ID NO: 72)
DIQMTQSPSSLSASVGDRVTITCRASQWIGSQLSWYQQKPGKAPMLLIMW
SSSLQSGVPSRFSGSGSGTDFTLTISSLQPEDFATYYCAQGLRHPKTFGQ
GTKVEIKW.
DOM7h-14-65
(SEQ ID NO: 73)
DIQMTQSPSSLSASVGDRVTITCRASQWIGSQLSWYQQKPGKAPKLLIMW
RSALQSGVPSRFSGSGSGTDFTLTISSLQPEDFATYYCAQGLRHPKTFGQ
GTKVEIKW.
DOM7h-14-74
(SEQ ID NO: 74)
DIQMTQSPSSLSASVGDRVTITCRASQWIGSQLSWYQQKPGKAPKLLIMW
RSSLQSGVPSRFSGSGSGTDFTLTISSLQPEDFATYYCAQGLRHPKTYGK
GTKVENKW.
DOM7h-14-76
(SEQ ID NO: 75)
DIQMTQSPSSLSASVGDRVTITCRASQWIGSQLSWYQQKPGKAPKLLIMW
RSSLQSGVPSRFSGSGSGTDFTLTISSLQPEDFATYYCAQGLKHPKTYGQ
GTKVEIKW.
DOM7h-14-82
(SEQ ID NO: 76)
DIQMTQSPSSLSASVGDRVTITCRASQWIGSQLSWYQQKPGKAPKLLIMW
RSSLQSGVPSRFSGSGSGTDFTLTISSLQPEDFATYYCAQGMRHPKTFGQ
GTKVEIKW.
DOM7h-14-100
(SEQ ID NO: 77)
DIQMTQSPSSLSASVGDRVTITCRASQWIGSQLSWYQQKPGKAPKLLIMW
RSSLQSGVPSRFSGSGSGTDFTLTISSLQPEDFATYYCAQGLRHPKTYGQ
GTKVENKW.
DOM7h-14-101
(SEQ ID NO: 78)
DIQMTQSPSSLSASVGDRVTITCRASQWIGSQLSWYQQKPGKAPKLLIMW
RSALQNGVPSRFSGSGSGTDFTLTISSLQPEDFATYYCAQGLRHPKTFGQ
GTKVEIKW.
DOM7h-14-109
(SEQ ID NO: 79)
DIQMTQSPSSLFASVGDRVTITCRASQWIGSQLSWYQQKPGKAPKLLIMW
RSSLQSGVPSRFSGSGSGTDFTLTISSLQPEDFATYYCAQGLRKPKTFGQ
GTKVKIKW.
DOM7h-14-115
(SEQ ID NO: 80)
DIQMTQSPSSLSASVGDRVTITCRASQWIGSQLSWYQQKPGKAPKLLIMW
RSALQSGVPSRFSGSGSGTDFTLTISSLQPEDFATYYCAQGLRHPKTYGQ
GTKVEIKW.
DOM7h-14-116
(SEQ ID NO: 81)
DIQMTQSPSSLSASVGDRVTITCRASQWIGSQLSWYQQKPGKAPKLLIMW
RSALQSGVPSRFSGSGSGTDFTLTISSLQPEDFATYYCAQGLRYPKTFGQ
GTKVEIKW.
DOM7h-14-119
(SEQ ID NO: 82)
DIQMTQSPSSLSASVGDRVTITCRASQWIGSQLSWYQQKPGKAPKLLIMW
RSSLQSGVPSRFSGSGSGTDFTLTISSLQPEDFATYYCAQGLRHPKTYGQ
GTKVEIKR.
DOM7h-14-120
(SEQ ID NO: 83)
DIQMTQSPSSLSASVGDRVTITCRASQWIGSQLSWYQQKPGKAPKLLIMW
RSSLQSGVPSRFSGSGSGTDFTLTISSLQPEDFATYYCAQGLRHPKTYGQ
GTKVENKR.
DOM7h-14-121
(SEQ ID NO: 84)
DIQMTQSPSSLSASVGDRVTITCRASQWIGSQLSWYQQKPGKAPKLLIMW
RSALQSGVPSRFSGSGSGTDFTLTISSLQPEDFATYYCAQGLRHPKTFGQ
GTKVEIKR.
DOM7h-14-122
(SEQ ID NO: 85)
DIQMTQSPSSLSASVGDRVTITCRASQWIGSQLSWYQQKPGKAPKLLIMW
RSSLQSGVPSRFSGSGSGTDFTLTISSLQPEDFATYYCAQGLRHPKTYGK
GTKVEIKR.
DOM7h-14-123
(SEQ ID NO: 86)
DIQMTQSPSSLSASVGDRVTITCRASQWIGSQLSWYQQKPGKAPKLLIMW
RSSLQSGVPSRFSGSGSGTDFTLTISSLQPEDFATYYCAQGLRHPKTYGK
GTKVENKR.

TABLE 16
Nucleotide sequences of DOM7h-14-10 variants

DOM7h-14-56
(SEQ ID NO: 87)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTATGCTCCTGATCATGTGG
AGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAGACGTTCGGCCAA
GGGACCAAGGTGGAAATCAAATGG.
DOM7h-14-65
(SEQ ID NO: 88)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCGCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAGACGTTCGGCCAA
GGGACCAAGGTGGAAATCAAATGG.
DOM7h-14-74
(SEQ ID NO: 89)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAGACGTACGGCAAA
GGGACCAAGGTGGAAAACAAATGG.
DOM7h-14-76
(SEQ ID NO: 90)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAAGCATCCTAAGACGTACGGCCAA
GGGACCAAGGTGGAAATCAAATGG.
DOM7h-14-82
(SEQ ID NO: 91)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTATGAGGCATCCTAAGACGTTCGGCCAA
GGGACCAAGGTGGAAATCAAATGG.
DOM7h-14-100
(SEQ ID NO: 92)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGCGGCATCCTAAGACGTACGGCCAA
GGGACCAAGGTGGAAAACAAATGG.
DOM7h-14-101
(SEQ ID NO: 93)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCGCGTTACAAAATGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAGACGTTCGGCCAA
GGGACCAAGGTGGAAATCAAATGG.
DOM7h-14-109
(SEQ ID NO: 94)
GACATCCAGATGACCCAGTCTCCATCCTCCCTGTTTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGAAACCTAAGACTTTCGGCCAA
GGGACCAAGGTGAAAATCAAATGG.
DOM7h-14-115
(SEQ ID NO: 95)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCGCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAAACGTACGGCCAA
GGGACCAAGGTGGAAATCAAATGG.
DOM7h-14-116
(SEQ ID NO: 96)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCGCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGTATCCTAAGACGTTCGGCCAA
GGGACCAAGGTGGAAATCAAATGG.
DOM7h-14-119
(SEQ ID NO: 97)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGCGGCATCCTAAGACGTACGGCCAA
GGGACCAAGGTGGAAATCAAACGG.
DOM7h-14-120
(SEQ ID NO: 98)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGCGGCATCCTAAGACGTACGGCCAA
GGGACCAAGGTGGAAAACAAACGG.
DOM7h-14-121
(SEQ ID NO: 99)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCGCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAGACGTTCGGCCAA
GGGACCAAGGTGGAAATCAAACGG.
DOM7h-14-122
(SEQ ID NO: 100)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAGACGTACGGCAAA
GGGACCAAGGTGGAAATCAAACGG.
DOM7h-14-123
(SEQ ID NO: 101)
GACATCCAGATGACCCAGTCTCCATCCTCCCTGTCTGCATCTGTAGGAGA
CCGTGTCACCATCACTTGCCGGGCAAGTCAGTGGATTGGGTCTCAGTTAT
CTTGGTACCAGCAGAAACCAGGGAAAGCCCCTAAGCTCCTGATCATGTGG
CGTTCCTCGTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGCTCAGGGTTTGAGGCATCCTAAGACGTACGGCAAA
GGGACCAAGGTGGAAAACAAACGG.

TABLE OF SEQUENCES

| | SEQ ID NO: | |
|---|---|---|
| Description | Amino acid | Nucleic acid |
| DOM7h-14-10 | 1 | 6 |
| DOM7h-14-18 | 2 | 7 |
| DOM7h-14-19 | 3 | 8 |
| DOM7h-14-28 | 4 | 9 |
| DOM7h-14-36 | 5 | 10 |
| DOM7h-14/Exendin-4 fusion | 11 | 12 |
| DOM7h-14-10/Exendin-4 fusion | 13 | 14 |
| DOM7h-14-18/Exendin-4 fusion | 15 | 16 |
| DOM7h-14-19/Exendin-4 fusion | 17 | 18 |
| DOM7h14-10/G4SC-NCE fusion | 19 | 20 |
| DOM7h14-10/TVAAPSC fusion | 21 | 22 |
| DPK9 Vk dummy CDR1 | 23 | |
| DPK9 Vk dummy CDR2 | 24 | |
| DPK9 Vk dummy CDR3 | 25 | |
| DOM 7h-14 CDR1 | 26 | |
| DOM 7h-14 CDR2 | 27 | |
| DOM 7h-14 CDR3 | 28 | |
| DOM 7h-14-10 CDR1 | 29 | |
| DOM 7h-14-10 CDR2 | 30 | |
| DOM 7h-14-10 CDR3 | 31 | |
| DOM 7h-14-18 CDR1 | 32 | |
| DOM 7h-14-18 CDR2 | 33 | |
| DOM 7h-14-18 CDR3 | 34 | |
| DOM 7h-14-19 CDR1 | 35 | |
| DOM 7h-14-19 CDR2 | 36 | |
| DOM 7h-14-19 CDR3 | 37 | |
| DOM 7h-14-28 CRD1 | 38 | |
| DOM 7h-14-28 CRD2 | 39 | |
| DOM 7h-14-28 CRD3 | 40 | |
| DOM 7h-14-36 CRD1 | 41 | |
| DOM 7h-14-36 CRD2 | 42 | |
| DOM 7h-14-36 CRD3 | 43 | |
| Interferon alpha 2b | 44 | 45 |
| IFNα2b SOE fragment 5′ | | 46 |
| IFNα2b SOE fragment 3′ | | 47 |
| Vk SOE fragment 5′ | | 48 |
| Vk SOE fragment 3′ to also introduce a myc | | 49 |
| tag | | |
| IFNα2b SOE fragment 5′ flanking primer | | 50 |
| Vk SOE fragment 3′ to also introduce a myc | | 51 |
| tag flanking primer | | |
| Leader sequence | 52 | 53 |
| DMS7321 | 54 | 55 |
| (IFNα2b-DOM7h-14) + myc | | |
| DMS7321 | 56 | 57 |
| (IFNα2b-DOM7h-14) | | |
| DMS732 | 58 | 59 |
| (IFNα2b-DOM7h-14-10) + myc | | |
| DMS732 | 60 | 61 |
| (IFNα2b-DOM7h-14-10) | | |
| DMS7323 | 62 | 63 |
| (IFNα2b-DOM7h-14-18) + myc | | |
| DMS7323 | 64 | 65 |
| (IFNα2b-DOM7h-14-18) | | |
| DMS7324 | 66 | 67 |
| (IFNα2b-DOM7h-14-19) + myc | | |
| DMS7323 | 68 | 69 |
| (IFNα2b-DOM7h-14-19) | | |
| DOM7h-14 R108C | 70 | 71 |
| DOM7h-14-56 | 72 | 87 |
| DOM7h-14-65 | 73 | 88 |
| DOM7h-14-74 | 74 | 89 |
| DOM7h-14-76 | 75 | 90 |
| DOM7h-14-82 | 76 | 91 |
| DOM7h-14-100 | 77 | 92 |
| DOM7h-14-101 | 78 | 93 |
| DOM7h-14-109 | 79 | 94 |
| DOM7h-14-115 | 80 | 95 |
| DOM7h-14-116 | 81 | 96 |
| DOM7h-14-119 | 82 | 97 |
| DOM7h-14-120 | 83 | 98 |
| DOM7h-14-121 | 84 | 99 |
| DOM7h-14-122 | 85 | 100 |
| DOM7h-14-123 | 86 | 101 |

1. An anti-serum albumin (SA) immunoglobulin single variable domain selected from the group consisting of: DOM7h-14-56 (SEQ ID NO: 72), DOM7h-14-65 (SEQ ID NO: 73), DOM7h-14-74 (SEQ ID NO: 74), DOM7h-14-76 (SEQ ID NO: 75), DOM7h-14-82 (SEQ ID NO: 76), DOM7h-14-100 (SEQ ID NO: 77), DOM7h-14-101 (SEQ ID NO: 78), DOM7h-14-109 (SEQ ID NO: 79), DOM7h-14-115 (SEQ ID NO: 80), DOM7h-14-116 (SEQ ID NO: 81), DOM7h-14-119 (SEQ ID NO: 82), DOM7h-14-120 (SEQ ID NO: 83), DOM7h-14-121 (SEQ ID NO: 84), DOM7h-14-122 (SEQ ID NO: 85) and DOM7h-14-123 (SEQ ID NO: 86).

2. A multispecific ligand comprising the anti-SA single variable domain of claim 1, and a binding moiety that specifically binds a target antigen other than SA.

3. The anti-SA single variable domain of claim 1, wherein the variable domain is conjugated to an NCE drug.

4. A fusion protein comprising a polypeptide or peptide drug fused to the single variable domain according to claim 1.

5. A composition comprising the variable domain of claim 1, and a pharmaceutically acceptable diluent, carrier, excipient or vehicle.

6. A nucleic acid comprising a nucleotide sequence encoding the single variable domain according to claim 1.

7. A nucleic acid comprising a nucleotide sequence that is at least 80% identical to a nucleotide sequence selected from the group consisting of: SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, and SEQ ID NO: 101.

8. A vector comprising the nucleic acid of claim 6.

9. An isolated host cell comprising the vector of claim 8.

10. A method of treating or preventing a disease or disorder in a patient, comprising administering at least one dose of the composition according to claim 5, to said patient.

11. A nucleic acid comprising a nucleotide sequence encoding the multispecific ligand of claim 2.

12. A vector comprising the nucleic acid of claim 11.

13. An isolated host cell comprising the vector of claim 12.

14. A nucleic acid comprising a nucleotide sequence encoding the fusion protein of claim 4.

15. A vector comprising the nucleic acid of claim 14.

16. An isolated host cell comprising the vector of claim 15.

17. A composition comprising the multispecific ligand of claim 2, and a pharmaceutically acceptable diluent, carrier, excipient or vehicle.

18. A composition comprising the fusion protein of claim 4, and a pharmaceutically acceptable diluent, carrier, excipient or vehicle.

19. A method of treating or preventing a disease or disorder in a patient, comprising administering at least one dose of the composition according to claim 17, to said patient.

20. A method of treating or preventing a disease or disorder in a patient, comprising administering at least one dose of the composition according to claim 18, to said patient.


SEQUENCE LISTING (101 sequences)

SEQ ID NO: 1 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 2 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLMKPMTFGQGTKVEIKR

SEQ ID NO: 3 - protein, 108 aa, Artificial Sequence
  Amino acid sequence identified using molecular biology techniques.
DIQMTQSPSSLSASVGDRVTISCRASQWIGSQLSWYQQKPGEAPKLLIMWRSSLQSGVPSRFSGSGSGTDFTLTISSLQPEDFATYYCAQGAALPRTFGQGTKVEIKR

SEQ ID NO: 4 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGAALPKTFGQGTKVEIKR

SEQ ID NO: 5 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGFKKPRTFGQGTKVEIKR

SEQ ID NO: 6 - DNA, 324 bp, Artificial Sequence
  Nucleotide sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 7 - DNA, 324 bp, Artificial Sequence
  Nucleotide sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtcttatgaagcctatgacgttcggccaagggaccaaggtggaaatcaaacgg

SEQ ID NO: 8 - DNA, 324 bp, Artificial Sequence
  Nucleotide sequence identified using molecular biology techniques.
gacatccagatgacccagtctccatcctccctgtctgcatctgtaggagaccgtgtcaccatctcttgccgggcaagtcagtggattgggtctcagttatcttggtaccagcagaaaccaggggaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 9 - DNA, 324 bp, Artificial Sequence
  Nucleotide sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacatactactgtgctcagggtgcggcgttgcctaagacgttcggccaagggaccaaggtggaaatcaaacgg

SEQ ID NO: 10 - DNA, 324 bp, Artificial Sequence
  Nucleotide sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggttttaagaagcctcggacgttcggccaagggaccaaggtggaaatcaaacgg

SEQ ID NO: 11 - protein, 163 aa, Artificial Sequence
  Amino acid sequence identified using molecular biology techniques.
HGEGTFTSDLSKQMEEEAVRLFIEWLKNGGPSSGAPPPSGGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKR

SEQ ID NO: 12 - DNA, 489 bp, Artificial Sequence
  Nucleotide sequence identified using molecular biology techniques.
catggtgaaggaacatttaccagtgacttgtcaaaacagatggaagaggaggcagtgcggttatttattgagtggcttaagaacggaggaccaagtagcggggcacctccgccatcgggtggtggaggcggttcaggcggaggtggcagcggcggtggcgggtcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 13 - protein, 163 aa, Artificial Sequence
  Amino acid sequence identified using molecular biology techniques.
HGEGTFTSDLSKQMEEEAVRLFIEWLKNGGPSSGAPPPSGGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 14 - DNA, 489 bp, Artificial Sequence
  Nucleotide sequence identified using molecular biology techniques.
catggtgaaggaacatttaccagtgacttgtcaaaacagatggaagaggaggcagtgcggttatttattgagtggcttaagaacggaggaccaagtagcggggcacctccgccatcgggtggtggaggcggttcaggcggaggtggcagcggcggtggcgggtcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 15 - protein, 163 aa, Artificial Sequence
  Amino acid sequence identified using molecular biology techniques.
HGEGTFTSDLSKQMEEEAVRLFIEWLKNGGPSSGAPPPSGGGGGSGGGGSGGGGSDIQMTQSPSSLSASVGDRVTITCRASQWIGSQLSWYQQKPGKAPKLLIMWRSSLQSGVPSRFSGSGSGTDFTLTISSLQPEDFATYYCAQGLMKPMTFGQGTKVEIKR

SEQ ID NO: 16 - DNA, 489 bp, Artificial Sequence
  Nucleotide sequence identified using molecular biology techniques.
catggtgaaggaacatttaccagtgacttgtcaaaacagatggaagaggaggcagtgcggttatttattgagtggcttaagaacggaggaccaagtagcggggcacctccgccatcgggtggtggaggcggttcaggcggaggtggcagcggcggtggcgggtcggacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtcttatgaagcctatgacgttcggccaagggaccaaggtggaaatcaaacgg

SEQ ID NO: 17 - protein, 163 aa, Artificial Sequence
  Amino acid sequence identified using molecular biology techniques.
HGEGTFTSDLSKQMEEEAVRLFIEWLKNGGPSSGAPPPSGGGGGSGGGGSGGGGSDIQMTQSPSSLSASVGDRVTISCRASQWIGSQLSWYQQKPGEAPKLLIMWRSSLQSGVPSRFSGSGSGTDFTLTISSLQPEDFATYYCAQGAALPRTFGQGTKVEIKR

SEQ ID NO: 18 - DNA, 489 bp, Artificial Sequence
  Nucleic acid sequence identified using molecular biology techniques.
catggtgaaggaacatttaccagtgacttgtcaaaacagatggaagaggaggcagtgcggttatttattgagtggcttaagaacggaggaccaagtagcggggcacctccgccatcgggtggtggaggcggttcaggcggaggtggcagcggcggtggcgggtcggacatccagatgacccagtctccatcctccctgtctgcatctgtaggagaccgtgtcaccatctcttgccgggcaagtcagtggattgggtctcagttatcttggtaccagcagaaaccaggggaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 19 - protein, 114 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKRGGGGSC

SEQ ID NO: 20 - DNA, 345 bp, Artificial Sequence
  Nucleotide sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacggggtggcggagggggttcctgt

SEQ ID NO: 21 - protein, 115 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKRTVAAPSC

SEQ ID NO: 22 - DNA, 345 bp, Artificial Sequence
  Nucleotide sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacggaccgtcgctgctccatcttgt

SEQ ID NO: 23 - protein, 9 aa, Artificial Sequence
  CDR 1 Amino acid sequence identified using molecular biology techniques.
SQSISSYLN

SEQ ID NO: 24 - protein, 8 aa, Artificial Sequence
  CDR 2 Amino acid sequence identified using molecular biology techniques.
YAASSLQS

SEQ ID NO: 25 - protein, 9 aa, Artificial Sequence
  CDR 3 Amino acid sequence identified using molecular biology techniques.
QQSYSTPNT

SEQ ID NO: 26 - protein, 9 aa, Artificial Sequence
  CDR 1 Amino acid sequence identified using molecular biology techniques.
SQWIGSQLS

SEQ ID NO: 27 - protein, 8 aa, Artificial Sequence
  CDR 2 Amino acid sequence identified using molecular biology techniques.
MWRSSLQS

SEQ ID NO: 28 - protein, 9 aa, Artificial Sequence
  CDR 3 Amino acid sequence identified using molecular biology techniques.
AQGAALPRT

SEQ ID NO: 29 - protein, 9 aa, Artificial Sequence
  CDR 1 Amino acid sequence identified using molecular biology techniques.
SQWIGSQLS

SEQ ID NO: 30 - protein, 8 aa, Artificial Sequence
  CDR 2 Amino acid sequence identified using molecular biology techniques.
MWRSSLQS

SEQ ID NO: 31 - protein, 9 aa, Artificial Sequence
  CDR 3 Amino acid sequence identified using molecular biology techniques.
AQGLRHPKT

SEQ ID NO: 32 - protein, 9 aa, Artificial Sequence
  CDR 1 Amino acid sequence identified using molecular biology techniques.
SQWIGSQLS

SEQ ID NO: 33 - protein, 8 aa, Artificial Sequence
  CDR 2 Amino acid sequence identified using molecular biology techniques.
MWRSSLQS

SEQ ID NO: 34 - protein, 9 aa, Artificial Sequence
  CDR 3 Amino acid sequence identified using molecular biology techniques.
AQGLMKPMT

SEQ ID NO: 35 - protein, 9 aa, Artificial Sequence
  CDR 1 Amino acid sequence identified using molecular biology techniques.
SQWIGSQLS

SEQ ID NO: 36 - protein, 8 aa, Artificial Sequence
  CDR 2 Amino acid sequence identified using molecular biology techniques.
MWRSSLQS

SEQ ID NO: 37 - protein, 9 aa, Artificial Sequence
  CDR 3 Amino acid sequence identified using molecular biology techniques.
AQGAALPRT

SEQ ID NO: 38 - protein, 9 aa, Artificial Sequence
  CDR 1 Amino acid sequence identified using molecular biology techniques.
SQWIGSQLS

SEQ ID NO: 39 - protein, 8 aa, Artificial Sequence
  CDR 2 Amino acid sequence identified using molecular biology techniques.
MWRSSLQS

SEQ ID NO: 40 - protein, 9 aa, Artificial Sequence
  CDR 3 Amino acid sequence identified using molecular biology techniques.
AQGAALPKT

SEQ ID NO: 41 - protein, 9 aa, Artificial Sequence
  CDR 1 Amino acid sequence identified using molecular biology techniques.
SQWIGSQLS

SEQ ID NO: 42 - protein, 8 aa, Artificial Sequence
  CDR 2 Amino acid sequence identified using molecular biology techniques.
MWRSSLQS

SEQ ID NO: 43 - protein, 9 aa, Artificial Sequence
  CDR 3 Amino acid sequence identified using molecular biology techniques.
AQGFKKPRT

SEQ ID NO: 44 - protein, 165 aa, Artificial Sequence [sequence omitted]

SEQ ID NO: 45 - DNA, 495 bp, Artificial Sequence
  Interferon alpha 2b nucleotide sequence identified using molecular biology techniques.
tgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaa

SEQ ID NO: 46 - DNA, 25 bp, Artificial Sequence
  Nucleic acid sequence identified using molecular biology techniques.
gcccggatccaccggctgtgatctg

SEQ ID NO: 47 - DNA, 30 bp, Artificial Sequence
  Nucleic acid sequence identified using molecular biology techniques.
ggaggatggagactgggtcatctggatgtc

SEQ ID NO: 48 - DNA, 30 bp, Artificial Sequence
  Nucleic acid sequence identified using molecular biology techniques.
gacatccagatgacccagtctccatcctcc

SEQ ID NO: 49 - DNA, 82 bp, Artificial Sequence
  Nucleic acid sequence identified using molecular biology techniques.
gcgcaagcttttattaattcagatcctcttctgagatgagtttttgttctgcggccgcccgtttgatttccaccttggtccc

SEQ ID NO: 50 - DNA, 25 bp, Artificial Sequence
  Nucleic acid sequence identified using molecular biology techniques.
gcccggatccaccggctgtgatctg

SEQ ID NO: 51 - DNA, 82 bp, Artificial Sequence; identical to SEQ ID NO: 49
  Nucleic acid sequence identified using molecular biology techniques.

SEQ ID NO: 52 - protein, 20 aa, Artificial Sequence
  Amino acid sequence identified using molecular biology techniques.
METDTLLLWVLLLWVPGSTG

SEQ ID NO: 53 - DNA, 61 bp, Artificial Sequence
  Nucleotide sequence identified using molecular biology techniques.
atggagaccgacaccctgctgctgtgggtgctgctgctgtgggtgcccggatccaccgggc

SEQ ID NO: 54 - protein, 293 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSDIQMTQSPSSLSASVGDRVTITCRASQWIGSQLSWYQQKPGKAPKLLIMWRSSLQSGVPSRFSGSGSGTDFTLTISSLQPEDFATYYCAQGAALPRTFGQGTKVEIKRAAAEQKLISEEDLN

SEQ ID NO: 55 - DNA, 882 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgggcggccgcagaacaaaaactcatctcagaagaggatctgaattaa

SEQ ID NO: 56 - protein, 882 aa, Artificial Sequence
  Amino acid sequence identified using molecular biology techniques.
TGCGACTTGCCACAGACACATAGTTTGGGATCAAGAAGAACATTGATGTTATTAGCACAAATGCGTAGAATTTCTTTGTTCTCTTGTCTAAAGGACCGTCACGACTTCGGATTCCCTCAGGAAGAGTTTGGAAACCAATTCCAAAAAGCAGAAACTATTCCTGTCTTGCACGAAATGATCCAGCAAATATTCAATTTGTTTTCTACAAAGGACTCATCAGCCGCTTGGGATGAAACTCTGTTAGATAAATTCTACACTGAACTATATCAACAACTGAACGATCTAGAGGCTTGCGTTATTCAGGGTGTAGGAGTTACTGAAACTCCCCTAATGAAAGAAGATTCAATTCTAGCCGTTAGAAAATACTTTCAGCGTATCACATTGTATTTAAAGGAAAAGAAATACTCCCCATGTGCATGGGAGGTGGTTAGAGCAGAAATTATGAGGTCCTTCTCTCTTTCTACGAATTTGCAAGAATCTTTGAGATCTAAGGAAACCGTCGCTGCTCCATCTGACATCCAGATGACCCAGTCTCCATCCTCCCTGTCTGCATCTGTAGGAGACCGTGTCACCATCACTTGCCGGGCAAGTCAGTGGATTGGGTCTCAGTTATCTTGGTACCAGCAGAAACCAGGGAAAGCCCCTAAGCTCCTGATCATGTGGCGTTCCTCGTTGCAAAGTGGGGTCCCATCACGTTTCAGTGGCAGTGGATCTGGGACAGATTTCACTCTCACCATCAGCAGTCTGCAACCTGAAGATTTTGCTACGTACTACTGTGCTCAGGGTGCGGCGTTGCCTAGGACGTTCGGCCAAGGGACCAAGGTGGAAATCAAACGGGCGGCCGCAGAACAAAAACTCATCTCAGAAGAGGATCTGAATTAA

SEQ ID NO: 57 - DNA, 837 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 58 - protein, 293 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSDIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKRAAAEQKLISEEDLN

SEQ ID NO: 59 - DNA, 882 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgggcggccgcagaacaaaaactcatctcagaagaggatctgaattaa

SEQ ID NO: 60 - protein, 279 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSDIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKR

SEQ ID NO: 61 - DNA, 837 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 62 - protein, 293 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSDIQMTQSPSSLSASVGDRVTITCRASQWIGSQLSWYQQKPGKAPKLLIMWRSSLQSGVPSRFSGSGSGTDFTLTISSLQPEDFATYYCAQGLMKPMTFGQGTKVEIKRAAAEQKLISEEDLN

SEQ ID NO: 63 - DNA, 882 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtcttatgaagcctatgacgttcggccaagggaccaaggtggaaatcaaacgggcggccgcagaacaaaaactcatctcagaagaggatctgaattaa

SEQ ID NO: 64 - protein, 279 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSDIQMTQSPSSLSASVGDRVTITCRASQWIGSQLSWYQQKPGKAPKLLIMWRSSLQSGVPSRFSGSGSGTDFTLTISSLQPEDFATYYCAQGLMKPMTFGQGTKVEIKR

SEQ ID NO: 65 - DNA, 837 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtcttatgaagcctatgacgttcggccaagggaccaaggtggaaatcaaacgg

SEQ ID NO: 66 - protein, 293 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSDIQMTQSPSSLSASVGDRVTISCRASQWIGSQLSWYQQKPGEAPKLLIMWRSSLQSGVPSRFSGSGSGTDFTLTISSLQPEDFATYYCAQGAALPRTFGQGTKVEIKRAAAEQKLISEEDLN

SEQ ID NO: 67 - DNA, 882 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgacatccagatgacccagtctccatcctccctgtctgcatctgtaggagaccgtgtcaccatctcttgccgggcaagtcagtggattgggtctcagttatcttggtaccagcagaaaccaggggaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgggcggccgcagaacaaaaactcatctcagaagaggatctgaattaa

SEQ ID NO: 68 - protein, 279 aa, Artificial Sequence
  Amino acid sequence identified using molecular biology techniques.
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKETVAAPSDIQMTQSPSSLSASVGDRVTISCRASQWIGSQLSWYQQKPGEAPKLLIMWRSSLQSGVPSRFSGSGSGTDFTLTISSLQPEDFATYYCAQGAALPRTFGQGTKVEIKR

SEQ ID NO: 69 - DNA, 837 bp, Artificial Sequence
  Nucleic acid sequence identified using molecular biology techniques.
tgcgacttgccacagacacatagtttgggatcaagaagaacattgatgttattagcacaaatgcgtagaatttctttgttctcttgtctaaaggaccgtcacgacttcggattccctcaggaagagtttggaaaccaattccaaaaagcagaaactattcctgtcttgcacgaaatgatccagcaaatattcaatttgttttctacaaaggactcatcagccgcttgggatgaaactctgttagataaattctacactgaactatatcaacaactgaacgatctagaggcttgcgttattcagggtgtaggagttactgaaactcccctaatgaaagaagattcaattctagccgttagaaaatactttcagcgtatcacattgtatttaaaggaaaagaaatactccccatgtgcatgggaggtggttagagcagaaattatgaggtccttctctctttctacgaatttgcaagaatctttgagatctaaggaaaccgtcgctgctccatctgacatccagatgacccagtctccatcctccctgtctgcatctgtaggagaccgtgtcaccatctcttgccgggcaagtcagtggattgggtctcagttatcttggtaccagcagaaaccaggggaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtgcggcgttgcctaggacgttcggccaagggaccaaggtggaaatcaaacgg

SEQ ID NO: 70 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTFGQGTKVEIKC

SEQ ID NO: 71 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaatgc

SEQ ID NO: 72 - protein, 108 aa, Artificial Sequence
  Amino acid sequence identified using molecular biology techniques.
DIQMTQSPSSLSASVGDRVTITCRASQWIGSQLSWYQQKPGKAPMLLIMWSSSLQSGVPSRFSGSGSGTDFTLTISSLQPEDFATYYCAQGLRHPKTFGQGTKVEIKW

SEQ ID NO: 73 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSALQSGVPSRFSGSGSGTDFTLTISSLQPEDFATYYCAQGLRHPKTFGQGTKVEIKW

SEQ ID NO: 74 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTYGKGTKVENKW

SEQ ID NO: 75 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLKHPKTYGQGTKVEIKW

SEQ ID NO: 76 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGMRHPKTFGQGTKVEIKW

SEQ ID NO: 77 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTYGQGTKVENKW

SEQ ID NO: 78 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSALQNGVPSRFSGSGSGTDFTLTISSLQPEDFATYYCAQGLRHPKTFGQGTKVEIKW

SEQ ID NO: 79 - protein, 108 aa, Artificial Sequence
  Amino acid sequence identified using molecular biology techniques.
DIQMTQSPSSLFASVGDRVTITCRASQWIGSQLSWYQQKPGKAPKLLIMWRSSLQSGVPSRFSGSGSGTDFTLTISSLQPEDFATYYCAQGLRKPKTFGQGTKVKIKW

SEQ ID NO: 80 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSALQSGVPSRFSGSGSGTDFTLTISSLQPEDFATYYCAQGLRHPKTYGQGTKVEIKW

SEQ ID NO: 81 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSALQSGVPSRFSGSGSGTDFTLTISSLQPEDFATYYCAQGLRYPKTFGQGTKVEIKW

SEQ ID NO: 82 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTYGQGTKVEIKR

SEQ ID NO: 83 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTYGQGTKVENKR

SEQ ID NO: 84 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSALQSGVPSRFSGSGSGTDFTLTISSLQPEDFATYYCAQGLRHPKTFGQGTKVEIKR

SEQ ID NO: 85 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTYGKGTKVEIKR

SEQ ID NO: 86 - protein, 108 aa, Artificial Sequence
  Amino acid sequence identified using molecular biology techniques.
DIQMTQSPSSLSASVGDRVTITCRASQWIGSQLSWYQQKPGKAPKLLIMWRSSLQSGVPSRFSGSGSGTDFTLTISSLQPEDFATYYCAQGLRHPKTYGKGTKVENKR

SEQ ID NO: 87 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctatgctcctgatcatgtggagttcctcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaatgg

SEQ ID NO: 88 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttccgcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaatgg

SEQ ID NO: 89 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgtacggcaaagggaccaaggtggaaaacaaatgg

SEQ ID NO: 90 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaagcatcctaagacgtacggccaagggaccaaggtggaaatcaaatgg

SEQ ID NO: 91 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtatgaggcatcctaagacgttcggccaagggaccaaggtggaaatcaaatgg

SEQ ID NO: 92 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgcggcatcctaagacgtacggccaagggaccaaggtggaaaacaaatgg

SEQ ID NO: 93 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttccgcgttacaaaatggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaatgg

SEQ ID NO: 94 - DNA, 324 bp, Artificial Sequence
  Nucleic acid sequence identified using molecular biology techniques.
gacatccagatgacccagtctccatcctccctgtttgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggaaacctaagactttcggccaagggaccaaggtgaaaatcaaatgg

SEQ ID NO: 95 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttccgcgttgcaaagtggggtcccatcacgtttcagtggcagtggatctgggacagatttcactctcaccatcagcagtctgcaacctgaagattttgctacgtactactgtgctcagggtttgaggcatcctaaaacgtacggccaagggaccaaggtggaaatcaaatgg

SEQ ID NO: 96 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttccgcgttgcaaagtggggtcccatcacgtttcagtggcagtggatctgggacagatttcactctcaccatcagcagtctgcaacctgaagattttgctacgtactactgtgctcagggtttgaggtatcctaagacgttcggccaagggaccaaggtggaaatcaaatgg

SEQ ID NO: 97 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgcggcatcctaagacgtacggccaagggaccaaggtggaaatcaaacgg

SEQ ID NO: 98 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgcggcatcctaagacgtacggccaagggaccaaggtggaaaacaaacgg

SEQ ID NO: 99 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttccgcgttgcaaagtggggtcccatcacgtttcagtggcagtggatctgggacagatttcactctcaccatcagcagtctgcaacctgaagattttgctacgtactactgtgctcagggtttgaggcatcctaagacgttcggccaagggaccaaggtggaaatcaaacgg

SEQ ID NO: 100 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgtacggcaaagggaccaaggtggaaatcaaacgg

SEQ ID NO: 101 - DNA, 324 bp, Artificial Sequence
  Nucleic acid sequence identified using molecular biology techniques.
gacatccagatgacccagtctccatcctccctgtctgcatctgtaggagaccgtgtcaccatcacttgccgggcaagtcagtggattgggtctcagttatcttggtaccagcagaaaccagggaaagcccctaagctcctgatcatgtggcgttcctcgttgcaaagtggggtcccatcacgtttcagtggcagtggatctgggacagatttcactctcaccatcagcagtctgcaacctgaagattttgctacgtactactgtgctcagggtttgaggcatcctaagacgtacggcaaagggaccaaggtggaaaacaaacgg